(19) United States
(12) Patent Application Publication
(10) Pub. No.: US 2026/0209717 A1
(43) Pub. Date: Jul. 23, 2026

(54) CELL CRYOPRESERVATION METHOD AND USE THEREOF

(71) Applicants: SUZHOU GRIT BIOTECHNOLOGY CO., LTD., Shanghai (CN); ZHUHAI TUOYU BIOTECHNOLOGY CO., LTD., Shanghai (CN); SHANGHAI GRIT BIOTECHNOLOGY CO., LTD., Shanghai (CN); SHENZHEN GRIT BIOTECHNOLOGY CO., LTD., Shanghai (CN)

(72) Inventors: Shuxia ZHOU, Shanghai (CN); Yarong LIU, Shanghai (CN); Zixiao SHI, Shanghai (CN)

(21) Appl. No.: 19/139,451

(22) PCT Filed: Dec. 19, 2023
(86) PCT No.: PCT/CN2023/139676

§ 371 Date: Jun. 16, 2025

(30) Foreign Application Priority Data
Dec. 20, 2022 (WO) PCT/CN2022/140245
Apr. 24, 2023 (WO) PCT/CN2023/090354

(51) Int. Cl. C12N 5/00

(52) U.S. Cl. CPC C12N 5/562

(57) ABSTRACT

The invention belongs to the field of biomedicine, and specifically provides a cell cryopreservation method and uses thereof. The cryopreservation method comprises programmed cooling steps, so that cryopreserved samples have a more stable cryopreservation effect.

### TECHNICAL FIELD

The invention belongs to the field of biomedicine, and specifically relates to a cell cryopreservation method and uses thereof.

### BACKGROUND

Immune cell cryopreservation is a reliable and economical method for long-term preservation of immune cells. The optimization or advance of immune cell cryopreservation technology is of great significance to the basic research and clinical application of immune cells.

However, there are shortcomings in the default cryopreservation steps of existing cryopreservation equipment, which make cryopreserved cells prone to intracellular crystallization during the cooling process, making it difficult for cells to survive after recovery. Therefore, there is an urgent need in this field for an effective cell cryopreservation method so that the cells have excellent stability during the cryopreservation process.

### CONTENTS OF THE INVENTION

In view of the technical problem in existing techniques that cells are not easy to survive after cryopreservation and recovery, the present invention provides a cryopreservation method.

In one aspect, the present invention provides a cryopreservation method, which comprises programmed cooling steps. For example, through a gradient cooling program, a cryopreservation system is cooled down smoothly. This cryopreservation method comprises:
(1) Reduce the overall temperature of the cryopreservation system to about 4 to 15 degrees Celsius,
(2) First reduce the temperature to about −2~−6 degrees Celsius at a rate of about 0.5~1.5 degrees Celsius/minute;
(3) Then reduce the temperature to −50~−60 degrees Celsius at a rate of about 15~25 degrees Celsius/minute;
(4) Then increase the temperature to −15~−25 degrees Celsius at a rate of about 8~15 degrees Celsius/minute;
(5) Then maintain the end temperature of the previous step for about 2~5 minutes;
(6) Then reduce the temperature to about −35~−45 degrees Celsius at a rate of about 0.5~1.5 degrees Celsius/minute;
(7) Then maintain the end temperature of the previous step for about 2~5 minutes;
(8) Finally, reduce the temperature to about −80~−90 degrees Celsius at a rate of about 8~15 degrees Celsius/minute.

In another aspect, the present invention provides a type of cells, which is cryopreserved by the cryopreservation method of the present invention.

In another aspect, the present invention provides a composition, which is cryopreserved by the cryopreservation method of the present invention.

In another aspect, the present invention provides a kit, which contains cells and/or compositions cryopreserved by the method of the present invention.

In another aspect, the invention provides a method of affecting cell growth, comprising administering cells of the invention and/or compositions of the invention. For example, the method comprises reducing the growth of tumor cells.

In another aspect, the invention provides the use of the cells of the invention and/or the compositions of the invention for the preparation of a medicament for the prevention and/or treatment of diseases and/or symptoms.

Compared with existing techniques, the beneficial effects of the present invention can be at least as follows: (1) During the cryopreservation process of the present invention, the cooling curve of the sample is stable; (2) Intracellular crystallization can be less likely to occur in the cell sample during the cryopreservation process of the present invention; (3) The cryopreserved cells of the present invention can achieve improved cell viability, optimized cell subpopulation ratio and/or improved cell killing ability after being recovered by commonly used recovery methods. The method of the present invention is used to cryopreserve immune cells, and can bring ideal application prospects to research and development and clinical work related to cell therapy storage.

### DESCRIPTION OF THE DRAWINGS

The features and advantages of the invention to which the present invention relates can be better understood by reference to the exemplary embodiments described in detail below and the accompanying drawings. A brief description of the drawings is as follows:

FIG. 1 shows the cooling curve of cryopreservation program 1 of the present invention.

FIG. 2 shows the cell counting results obtained from each cooling program.

FIG. 3 shows the flow cytometric detection results of cells obtained from each cooling program.

FIG. 4 shows the results of promoting tumor cell killing of cells obtained from each cooling program.

FIG. 5 shows the results of promoting cytokine secretion of cells obtained from each cooling program.

FIG. 6 shows the viability and yield results of using cryopreservation program 1 of the present invention for PBMC cell cryopreservation.

FIG. 7 shows the TIL activating results of using cryopreservation program 1 of the present invention for PBMC cell cryopreservation.

FIG. 8 shows the viability and yield results of using cryopreservation program 1 of the present invention for NK cell cryopreservation.

FIG. 9 shows the viability results of using cryopreservation program 1 of the present invention for TCR-T cell cryopreservation.

FIG. 10 shows the tumor cell killing results of using cryopreservation program 1 of the present invention for TCR-T cells.

FIG. 11 shows the cooling curve of cryopreservation program 2 of the present invention.

FIG. 12 shows the cell counting results obtained from cryopreservation program 2 of the present invention.

FIG. 13 shows the flow cytometric detection results of cells obtained from cryopreservation program 2 of the present invention.

FIG. 14 shows the results of promoting tumor cell killing of cells obtained from cryopreservation program 2 of the present invention.

FIG. 15 shows the results of promoting cytokine secretion of cells obtained from cryopreservation program 2 of the present invention.

FIG. 16 shows the cooling curve of the cryopreservation program 1-1 of the present invention.

FIG. 17 shows the cooling curve of cryopreservation program 1-2 of the present invention.

FIG. 18 shows the cooling curve of cryopreservation program 1-3 of the present invention.

FIG. 19 shows the cooling curve of cryopreservation program 2-1 of the present invention.

FIG. 20 shows the cooling curve of cryopreservation program 2-2 of the present invention.

### DETAILED EMBODIMENTS

The embodiments of the present invention will be described below with specific examples. Those skilled in the art can easily understand other advantages and effects of the present invention from the content disclosed in this specification.

### Definition of Terms

In the present invention, the term “immune cells” generally refers to cells involved in innate and adaptive immune responses. For example, immune cells may include, but are not limited to, lymphocytes (such as T cells (including thymocytes) and B cells), natural killer (NK) cells, NKT cells, macrophages, monocytes, eosinophils, basophils, neutrophils, dendritic cells, and mast cells. In some embodiments, T cells include, for example, CD4+ T cells, CD8+ T cells (also known as cytotoxic T cells or CTLs), regulatory T cells (Tregs), Th1 cells, Th2 cells, Th17 cells, αβ T cells, and/or γδ T cells. In some embodiments, immune cells also include modified immune effector cells, such as chimeric antigen receptor (CAR)-modified immune effector cells (e.g., CAR-T cells, CAR-NK cells), T cell receptors (TCR)-modified immune effector cells (such as TCR-T cells).

In the present invention, the term “chimeric antigen receptor (CAR)” generally refers to an engineered antigen receptor. For example, a CAR may comprise an extracellular antigen binding domain fused via a hinge and transmembrane domain to a cytoplasmic domain comprising a signaling domain. In some embodiments, a CAR extracellular domain can bind to an antigen expressed by a target cell in an MHC-independent manner, resulting in activation and proliferation of the cell. In some embodiments, the extracellular domain of the CAR can recognize a tag fused to an antibody or antigen-binding fragment thereof. For example, a single CAR construct can be made to target multiple different antigens by substituting one antibody for another. In some embodiments, the extracellular domain of the CAR may comprise an antigen-binding fragment derived from an antibody. The antigen-binding domains of the invention may include, for example, scFv, antibodies, antigen-binding regions of antibodies, variable regions of heavy/light chains, and/or single-chain antibodies.

In the present invention, the term “T cell receptor (TCR)” generally refers to an engineered antigen receptor. For example, a TCR may comprise TCRα and/or TCRβ chains that have been isolated and cloned from a population of T cells that recognize a specific target antigen. For example, TCRα and/or TCRβ genes (i.e., TRAC and TRBC) can be cloned from T cell populations isolated from individuals with specific malignant tumors or from T cells that have been isolated from humanized mice immunized with specific tumor antigens or tumor cells. Engineered TCRs can recognize antigens through the same mechanism as their endogenous counterparts (e.g., by recognizing their cognate antigens presented in the context of major histocompatibility complex (MHC) proteins expressed on the target cell surface), thereby leading to activation and proliferation of TCR-engineered cells.

In the present invention, the term “encoding” generally refers to directly or indirectly inferring, from the structure or composition information of one molecule, according to basically determined rules, the structure or composition information of another type of molecule related to it. For example, from amino acid sequence, its nucleotide sequence can be inferred, for example based on the characteristics of deoxyribonucleic acid-transcribed complementary nucleic acids, including nucleic acids that can be translated into polypeptides. For example, deoxyribonucleic acid may encode RNA transcribed from the deoxyribonucleic acid. Deoxyribonucleic acid may similarly encode a polypeptide translated from the RNA transcribed from the deoxyribonucleic acid.

In the present invention, the term “NK cell” is also called “natural killer cell” and generally refers to a cell with large granules in the cytoplasm. NK cells are developed from bone marrow lymphoid stem cells and can differentiate and develop depending on the bone marrow or thymus microenvironment. In the present invention, the ratio of NK cells in TIL cells can be changed by the method of the present invention.

In the present invention, “CD4⁺ cells” usually refer to CD4-positive cells, such as T cells. The terms “CD4⁺ cells” and “CD4-positive cells” may be used synonymously. These cells may be identified by methods known in the art, such as by staining the cells with fluorescently labeled antibodies against CD4 and using fluorescence-activated cell sorting.

In the present invention, “CD8⁺ cells” usually refer to CD8-positive cells, which may be T cells, for example. The terms “CD8⁺ cells” and “CD8-positive cells” may be used synonymously. These cells may be identified by methods known in the art, such as by staining the cells with fluorescently labeled antibodies against CD8 and using fluorescence-activated cell sorting.

In the present invention, the term “tumor-infiltrating lymphocytes” or “TILs” generally refers to a population of cells originally obtained as white blood cells that have left the subject's bloodstream and migrated into the tumor. TILs may include but are not limited to CD8⁺ cytotoxic T cells (lymphocytes), Th1 and Th17 CD4⁺ T cells, natural killer cells, dendritic cells and M1 macrophages. TILs may include primary TILs and secondary TILs. “Primary TILs” can be those TIL cells obtained from a subject's tissue sample, and “secondary TILs” can be any of the expanded TIL populations in the present invention. In some embodiments, the tumor-infiltrating lymphocytes of the present invention may have not been isolated or purified, or may infiltrate tumor cells. For example, TIL in the present invention may refer to a TIL population.

In the present invention, the term “central memory cells” generally refers to cells with long-term memory and capable of receiving antigen restimulation. Central memory cells can have CD45RA⁻C CR7⁺ or CD45RO⁺ CD62L⁺ phenotype, and central memory cells can for example be identified by CD45RA⁻ and CCR7⁺ or CD45RO⁺ and CD62L⁺. Central memory cells can have a stronger ability to suppress tumor growth than ordinary cells.

In the present invention, the term “regulatory cells” generally refers to a subpopulation of cells that control autoimmune response in the body. Regulatory cells can have CD4⁺ CD25⁺ Foxp3⁺ phenotype, and regulatory cells can for example be identified by CD4⁺, CD25⁺ and Foxp3⁺. Regulatory cells may have the ability to inhibit the tumor growth-suppressing ability of cells.

In the present invention, the term “activated cells” generally refers to cells that have been activated to have the ability to suppress tumor growth. Activated cells can have CD28⁺, CD25⁺, or 41BB⁺ phenotype, and activated cells can for example be identified by CD 28⁺, CD 25⁺, or 41BB⁺. Activated cells can have the ability to suppress tumor growth.

In the present invention, the term “exhausted cells” generally refers to immune cells that are continuously stimulated by antigens and gradually lose their effector functions. For example, the function of exhausted cells may be reversible, or partially reversible. Exhausted cells can have PD1⁺, LAG3⁺, TIM3⁺, or CD39⁺ phenotype, and exhausted cells can for example be identified by PD1⁺, LAG3⁺, TIM3⁺, or CD39⁺. Exhausted cells can be characterized by reduced immune function.

In the present invention, the term “apoptotic cells” generally refers to immune cells undergoing programmed death. Apoptotic cells can have CD95⁺ caspass3⁺ cells' and/or CD95⁺ DR5⁺ phenotype, and apoptotic cells can for example be identified by CD95⁺ caspass3⁺ cells and/or CD95⁺ DR5⁺. Apoptotic cells can cause a decrease in cell number.

In the present invention, the term “tumor-specific cells” generally refers to cells that can specifically suppress tumor growth. Tumor-specific cells can possess CD103⁺ CD39⁺ phenotype, and tumor-specific cells can for example be identified by CD103⁺ and CD39⁺. Tumor-specific cells can have a more specific ability to suppress tumor growth than ordinary cells.

In the present invention, the term “stem cell-like cells” generally refers to a type of cells that may have the potential for self-proliferation and/or differentiation (stemness). Stem cell-like cells can have CD69⁻ CD39⁻ or TCF1⁺ phenotype, and stem cell-like cells can for example be identified by CD69⁻ CD39⁻ or TCF1⁺. Tumor-specific cells may have stronger and/or longer-term ability to suppress tumor growth than normal cells.

In the present invention, the term “cell subpopulation ratio” generally refers to the ratio of different cell subpopulations in immune cells or immune cell populations. For example, different cell subpopulations of the present invention have different immune activities and/or differentiation abilities. For example, cell subpopulations of the invention can be distinguished based on cell surface markers.

In the present invention, the term “in vivo” generally refers to events occurring within the body of a subject.

In the present invention, the term “in vitro” generally refers to events that occur outside the body of a subject.

In the present invention, the term “ex vivo” generally refers to events involving treatment or surgery on cells, tissues and/or organs that have been removed from the body of a subject. For example, the cells, tissues and/or organs can be returned to the subject's body through surgery or treatment.

In the present invention, the term “expansion” generally refers to a several-fold increase in the number of cells over a period of time. For example, the number of cells can be increased by at least about 3-fold (or 4, 5, 6, 7, 8, or 9 folds), for example, the number of cells can be increased by at least about 10-fold (or 20, 30, 40, 50, 60, 70, 80 or 90-fold), or, for example, the number of cells can be increased by at least about 100-fold. In the present invention, the term “expanded” generally means that the cells of the invention have undergone one or more of the above-mentioned expansions.

In the present invention, the term “improved immune cell properties” generally refers to immune cell properties that occur after the cells have been expanded and/or cultured. Improved immune cell properties may include changes in the number and/or proportion of cells, changes in secretory ability, changes in killing ability, or changes in expression ability, or any combination thereof. According to different evaluation criteria, the improvement in the present invention can be an increase or a decrease, such as an increase in killing ability and a decrease in cell exhaustion level.

In the present invention, the term “killing ability” generally refers to killing target cells by contacting the cells of the present invention with an effective amount of a substance. In one embodiment, the substance of the invention may be immune cells. Killing of the present invention may include killing cells by itself or by promoting CDC, apoptosis, ADCC, and/or phagocytosis of other cells or substances, or by a combination of two or more of these mechanisms.

In the present invention, the term “administering” generally refers to the delivery of a substance to a subject in need thereof by any route known in the art. Pharmaceutical carriers and preparations or compositions are also well known in the art. Routes of administration may include: intravenous, intramuscular, intradermal, subcutaneous, transdermal, mucosal, intratumoral and/or mucosal.

In the present invention, the term “kit” generally refers to two or more components packaged together in a container, receptacle or other container, one of which corresponds to a substance of the invention, for example, an immune cell.

In the present invention, the term “subject” generally refers to a cell or an animal, which may be a mammal, such as a human, a non-human primate (apes, gibbons, gorillas, chimpanzees, orangutans, macaques), a domestic animal (dogs and cats), a farm animal (poultry such as chickens and ducks, horses, cattle, goats, sheep, pigs) and a laboratory animal (mice, rats, rabbits, guinea pigs). Human subjects include fetal, neonatal, infant, adolescent, and adult subjects. Subjects include animal disease models, such as tumor animal models, and other animal models known to those skilled in the art.

In the present invention, the term “feeder” generally refers to cultured cells that grow in vitro and secret at least one factor to the culture medium, and that can be used to support the growth of another type of cells of interest. For example, feeder cells may include antigen-presenting cells.

In the present invention, the term “cell culture medium” generally refers to the nutrient solution in which cells, such as mammalian cells, are grown. The preparation of cell culture media is well known in the art. Typically, cell culture media includes buffers, salts, carbohydrates, amino acids, vitamins, and necessary trace elements. Cell culture media may or may not contain serum, peptone, and/or protein. Cell culture media can be supplemented with additional components or components of increased concentration, such as amino acids, salts, sugars, vitamins, hormones, growth factors, buffers, antibiotics, lipids, trace elements, etc., depending on the requirements of the cells to be cultured and/or desired cell culture parameters.

In the present invention, the term “pharmaceutical composition” or “pharmaceutical preparation” generally refers to a preparation that allows the biological activity of the active ingredient to be effective and does not contain additional components that are unacceptably toxic to the subject to whom the preparation is administered. Such preparations are sterile. “Pharmaceutically acceptable” excipients (carriers, additives) are those which can be reasonably administered to a subject mammal to provide an effective dose of the active ingredient employed.

In the present invention, the term tumor “fragments” generally refers to tumor fragments that are formed after tumor tissue is removed from the subject's body, which can be achieved by mechanical fragmentation, enzymatic digestion and/or other fragmentation methods

In the present invention, the term “composition” or “pharmaceutical composition” generally refers to mixtures of at least one type of cells and at least one and optionally more than one other pharmaceutically acceptable chemical components such as carriers, stabilizers, diluents, dispersing agents, suspending agents, thickeners and/or excipients.

In the present invention, the term “pharmaceutically acceptable carrier” generally refers to one or more non-toxic materials that do not interfere with the active ingredient. For example, a pharmaceutically acceptable carrier may not interfere with the biological activity of the active ingredient; for example, a pharmaceutically acceptable carrier may not interfere with the effectiveness of the biological activity possessed by the active ingredient. Such preparations may conventionally contain salts, buffers, preservatives, compatible carriers, and optionally other therapeutic agents. These pharmaceutically acceptable preparations may also contain compatible solid or liquid fillers, diluents or encapsulating substances suitable for administration to humans. Other contemplated carriers, excipients, and/or additives that may be used in the preparations described herein may include, for example, flavoring agents, antimicrobial agents, sweeteners, antioxidants, antistatic agents, lipids, protein excipients (such as serum albumin, gelatin, casein), salt-forming counterions (such as sodium), etc. These and other known pharmaceutical carriers, excipients and/or additives suitable for use in the preparations described herein are known in the art. In the present invention, “pharmaceutically acceptable carrier” can be understood as not including vectors in the form of nucleic acids used in genetic engineering.

In the present invention, the term “functionally active fragment” generally refers to a fragment that has a partial region of a full-length protein or nucleic acid, but retains or partially retains the biological activity or function of the full-length protein or nucleic acid. For example, a functionally active fragment may retain or partially retain the ability of the full-length protein to bind another molecule. For example, a functionally active fragment of the growth factor IL-2 may retain or partially retain the biologically active function of full-length IL-2 that causes cell proliferation.

In the present invention, the term “substantially simultaneously” generally means that immune cells can be in contact with more than two substances simultaneously within a period of time during the contact process, but is not limited to immune cells always being in contact simultaneously with more than two substances during the entire contact process. For example, substantially simultaneously means that for a period of time, immune cells can be in contact simultaneously with at least 10%, 20%, 30%, 40%, 50%, 60%, 70%, 75%, 80%, 85%, 90%, 95% of each substance among more than two substances.

In the present invention, the term “tumor” generally refers to any new pathological tissue proliferation. The tumors of the present invention may be benign or malignant. The tumors of the present invention may be solid or hematological. The term “tumor” may be one or more types selected from the following group: melanoma, ovarian cancer, cervical cancer, lung cancer, bladder cancer, breast cancer, head and neck cancer, pancreatic cancer, liver cancer, gastric cancer, colorectal cancer, and kidney cancer.

In the present invention, the term “tumor tissue” generally refers to tumors from a subject, including samples of any tissue of any solid tumor and/or non-solid tumor in the subject.

In the present invention, the term “number of immune cells” generally refers to the number of cells in the immune cells of the invention. In the present invention, the number of immune cells may refer to the number of cells in immune cell populations obtained at any stage of the present invention. For example, the number of immune cells may refer to the number of cells of the first immune cell population derived from tumor tissue, pleural effusion and/or peritoneal effusion without in vitro expansion. For example, the number of immune cells may refer to the number of cells of the second TIL population expanded in vitro in the first stage. For example, the number of TIL cells may refer to the number of cells of the third TIL population expanded in vitro in the second stage. For example, the number of immune cells may refer to the cell of immune cells finally obtained by any culture method of the present invention. In the present invention, the number of immune cells can be measured by methods commonly used in the art, including but not limited to manual cell counting with a hemocytometer and/or counting with an automatic cell counter.

In the present invention, the terms “about” and “approximately” generally refer to a statistically meaningful numerical range. Such a range may be within an order of magnitude of a given value or range, may include within 50%, may include within 20%, may include within 10%, and may include within 5%. Allowable variations encompassed by the terms “about” or “approximately” depend on the particular system being studied, and are readily understood by those of ordinary skill in the art.

In the present invention, the terms “more than”, “less than”, “at most” and “at least” include the stated number.

### DETAILED DESCRIPTION

### Cryopreservation Program One

In one aspect, the invention provides a cryopreservation method, comprising the following steps:
(1) Reduce the overall temperature of a cryopreservation system to about 4 to 15 degrees Celsius,
(2) First reduce the temperature to about −2~−6 degrees Celsius at a rate of about 0.5~1.5 degrees Celsius/minute;
(3) Then reduce the temperature to −50~−60 degrees Celsius at a rate of about 15~25 degrees Celsius/minute;
(4) Then increase the temperature to −15~−25 degrees Celsius at a rate of about 8~15 degrees Celsius/minute (for example, 8~12 degrees Celsius/minute);
(5) Then maintain the end temperature of the previous step for about 2~5 minutes;
(6) Then reduce the temperature to about −35~−45 degrees Celsius at a rate of about 0.5~1.5 degrees Celsius/minute;
(7) Then maintain the end temperature of the previous step for about 2~5 minutes;
(8) Finally, reduce the temperature to about −80~−90 degrees Celsius at a rate of about 8~15 degrees Celsius/minute.

In one aspect, the invention provides a cryopreservation method, comprising the following steps:
(1) Reduce the overall temperature of a cryopreservation system to about 4 to 15 degrees Celsius,
(2) First reduce the temperature to about −4 degrees Celsius at a rate of about 1 degrees Celsius/minute;
(3) Then reduce the temperature to −55 degrees Celsius at a rate of about 20 degrees Celsius/minute;
(4) Then increase the temperature to −20 degrees Celsius at a rate of about 10 degrees Celsius/minute;
(5) Then maintain the temperature at about −20 degrees Celsius for about 3 minutes;
(6) Then reduce the temperature to about −40 degrees Celsius at a rate of about 1 degrees Celsius/minute;
(7) Then maintain the temperature at about −40 degrees Celsius for about 3 minutes;
(8) Finally, reduce the temperature to about −90 degrees Celsius at a rate of about 10 degrees Celsius/minute.

In one aspect, the invention provides a cryopreservation method, comprising the following steps:
(1) Reduce the overall temperature of a cryopreservation system to about 4 to 15 degrees Celsius,
(2) First reduce the temperature to about −2 to −4 degrees Celsius at a rate of about 0.5 to 1.5 degrees Celsius/minute;
(3) Then reduce the temperature to −50 to −60 degrees Celsius at a rate of about 15 to 25 degrees Celsius/minute;
(4) Then increase the temperature to −18 to −20 degrees Celsius at a rate of about 8 to 15 degrees Celsius/minute (for example, 10 to 15 degrees Celsius/minute);
(5) Then maintain the end temperature of the previous step for about 2 to 5 minutes;
(6) Then reduce the temperature to about −35 to −45 degrees Celsius at a rate of about 0.5 to 1.5 degrees Celsius/minute;
(7) Then maintain the end temperature of the previous step for about 2 to 5 minutes;
(8) Finally, reduce the temperature to about −80 to −90 degrees Celsius at a rate of about 8 to 15 degrees Celsius/minute.

In one aspect, the invention provides a cryopreservation method, comprising the following steps:
(1) Reduce the overall temperature of a cryopreservation system to about 4 to 15 degrees Celsius, for example 4 degrees Celsius,
(2) First reduce the temperature to about −2 to −4 degrees Celsius at a rate of about 0.5 to 1.5 degrees Celsius/minute;
(3) Then reduce the temperature to −50 to −60 degrees Celsius at a rate of about 15 to 25 degrees Celsius/minute;
(4) Then increase the temperature to −18 to −20 degrees Celsius at a rate of about 8 to 15 degrees Celsius/minute (for example, 10 to 15 degrees Celsius/minute);
(5) Then maintain the end temperature of the previous step for about 2 to 5 minutes;
(6) Then reduce the temperature to about −35 to −45 degrees Celsius at a rate of about 0.5 to 1.5 degrees Celsius/minute;
(7) Then maintain the end temperature of the previous step for about 2 to 5 minutes;
(8) Finally, reduce the temperature to about −80 to −90 degrees Celsius at a rate of about 8 to 15 degrees Celsius/minute.

Among these, the cryopreservation system usually refers to placing the materials to be cryopreserved in a controlled cooling environment to form a system. For example, the controlled cooling environment can be a cooling compartment of cooling equipment, a cooling chamber, etc. For example, the method provided by the present invention uses steps to cause the ambient temperature of the cryopreservation system to increase or decrease at a set rate, or be maintained at a set temperature, so that the material to be cryopreserved can cool down gradually and pass through the crystallization nucleation point smoothly, avoiding damage to cells caused by intracellular crystallization. For example, the method provided by the present invention uses steps to cause the temperature in the chamber of the cooling instrument to increase or decrease at a set rate, or be maintained at a set temperature.

For example, the method may further comprise mixing a cryopreservation agent of a concentration of about 2.0% to 6.0% before cooling down the cryopreservation system. For example, the cryopreservation agent may comprise CS10. For example, the cryopreservation agent may comprise DMSO. For example, the cryopreservation agent is DMSO, and its concentration in the cryopreservation system is about 2.0% to 6.0%, or about 2.0% to 4.5%, or about 2.0% to 4.0%, or about 2.0% to 3.8%, or about 2.0% to 3.75%, or about 2.0% to 3.0%, or about 2.5% to 4.5%, or about 2.5% to 4.0%, or about 2.5% to 3.8%, or about 2.5% to 3.75%, or about 2.5% to 3.0%, or about 3.0% to 4.5%, or about 3.0% to 4.0%, or about 3.0% to 3.8%, or about 3.0% to 3.75%, Or about 3.75% to 4.5%, or about 3.8% to 4.5%, or about 3.75% to 4.0%, or about 3.8% to 4.0%, or about 4.0% to 4.5% (w/v). For example, the cryopreservation agent may comprise (w/v): 5~70%, preferably 10~50% of CS 10, 1-50%, preferably 2~25% 20% of human albumin injection. For example, the cryopreservation agent may comprise about 5%, about 10%, about 15%, about 20%, about 25%, about 30%, about 35%, about 40%, about 45%, about 50%, about 55%, about 60%, or about 70% (w/v) CS 10. For example, the cryopreservation agent may also comprise human albumin. For example, the cryopreservation agent may comprise about 1%, about 2%, about 3%, about 5%, about 10%, about 15%, about 20%, about 25%, about 30%, about 35%, about 40%, about 45%, about 50% (w/v) of 20% human albumin injection.

For example, the method may also comprise mixing a sample comprising cells with a culture medium before cooling the cryopreservation system, and the culture medium may comprise a commercial cell culture medium. For example, the culture medium of the present invention may comprise serum, or be serum-free. For example, cells can be suspended in cell culture medium at the following density: approximately 1×10⁵ cells/mL to approximately 2×10⁸ cells/mL, about 1×10⁵ cells/mL to approximately 1×10⁸ cells/mL, about 1×10⁶ cells/mL to approximately 1×10⁸ cells/mL, about 1×10⁷ cells/mL to approximately 1×10⁸ cells/mL, or about 4×10⁷ cells/mL to 1×10⁸ cells/mL. In some embodiments, cells are suspended in the cryopreservation solution at the following density: about 1×10⁵ cells/mL, about 5×10⁵ cells/mL, about 1×10⁶ cells/mL, about 2×10⁶ cells/mL, about 5×10⁶ cells/mL, about 1×10⁷ cells/mL, about 1.5×10⁷ cells/mL, about 2×10⁷ cells/mL, about 2.5×10⁷ cells/mL, about 3×10⁷ cells/mL, about 3.5×10⁷ cells/mL, about 4×10⁷ cells/mL, about 4.5×10⁷ cells/mL, about 5.0×10⁷ cells/mL, about 5.5×10⁷ cells/mL, about 6.0×10⁷ cells/mL, about 6.5×10⁷ cells/mL, about 7.0×10⁷ cells/mL, about 7.5×10⁷ cells/mL, about 8.0×10⁷ cells/mL, about 8.5×10⁷ cells/mL, about 9.0×10⁷ cells/mL, about 9.5×10⁷ cells/mL, about 1×10⁸ cells/mL, about 1.5×10⁸ cells/mL or approximately 2×10⁸ cells/mL. In certain embodiments, cells are suspended at a density of approximately 1.5×10⁷ cells/mL to approximately 6×10⁷ cells/mL in the cryopreservation solution. In certain embodiments, cells are suspended at a density of approximately 5×10⁶ cells/mL to approximately 150×10⁶ cells/mL in the cryopreservation solution. In certain embodiments, cells are suspended at a density of at least about 1×10⁷ cells/mL in the cryopreservation solution. In specific embodiments, cells are suspended at a density of at least about 5.0×10⁷ cells/mL in the cryopreservation solution. In some embodiments, the cells are viable cells. In some embodiments, cell density is determined by cell diameter.

For example, the method may also comprise storing the cryopreservation system at about −80 degrees Celsius or below after cooling down the cryopreservation system. For example, the method may also comprise storing the cryopreservation system in liquid nitrogen after cooling down the cryopreservation system. For example, the ambient temperature of the storage may be at a temperature from −196°° C. to −80° C.

For example, the cryopreservation system may comprise placing the sample in a cryovial. For example, the volume of the cryopreservation system is about 1 microliter to 1 liter. For example, the sample volume in the cryopreservation system can be about 1 microliter, about 10 microliters, about 100 microliters, about 1000 microliters, about 1.5 milliliters, about 2 milliliters, about 2.5 milliliters, about 3.5 milliliters, about 4 milliliters, about 5 milliliters, about 10 milliliters, or about 100 milliliters.

For example, the cryopreservation system may comprise cells. For example, the density of the cells in the cryopreservation system is approximately 1×10⁵ to 1×10⁹ cells/mL. For example, before cooling down the cryopreservation system, the sample comprising cells and the culture medium can be mixed at a volume ratio of about 1:0.1~1:50, preferably about 1:0.5~1:10. For example, before the cryopreservation system is cooled down, the sample comprising cells and the culture medium can be mixed at a volume ratio of about 1:0.1, about 1:0.2, about 1:0.5, about 1:1, about 1:2, about 1:5, about 1:10, about 1:20, or about 1:50. For example, the cell culture medium may be a basal culture medium known in the art. For example, the cell culture medium can be 1640 or DMEM.

For example, the cells may comprise immune cells. For example, immune cells enriched in samples of the present invention include CD4⁺ T cells or subsets thereof and/or CD8⁺ T cells or subsets thereof. For example, CD4⁺ cell subsets and/or CD8⁺ cell subsets are optionally selected from the following cells and combinations thereof: memory cells, central memory T (T_{CM}) cells, effector memory cells (T_{EM}), stem cell-like central memory (T_{SCM}) cells, T effector (T_{E}) cells, naive T (T_{N}) cells, regulatory T (T_{REG}) cells and/or helper T cells; or TH1 cells, TH2 cells, TH3 cells, TH17 cells, TH9 cells, TH22 cells, follicular helper T cells, α/β T cells and δ/γ T cells. For example, samples of the invention comprise primary T cells obtained from a subject. For example, samples of the invention include one or more subsets of T cells or other types of cells, such as the entire T cell population, CD4⁺ cells, CD8⁺ cells and their subsets. For example, samples of the invention include T cells isolated from a PBMC sample by negative selection for markers expressed on non-T cells, such as B cells, monocytes or other leukocytes, such as CD14. In some aspects, CD4⁺ or CD8⁺ selection steps are used to isolate CD4⁺ helper T cells and CD8⁺ cytotoxic T cells. CD4⁺ and CD8⁺ populations may be further grouped into subsets by positive or negative selection for markers expressed on or to a relatively higher degree on one or more naive T cell, memory T cell and/or effector T cell subsets. For example, biological samples include T cells and/or engineered T cells. For example, engineered T cells include T cells that express recombinant molecules or exogenous molecules, which are optionally recombinant proteins, optionally recombinant receptors, which are optionally T cell receptors (TCR), chimeric receptors, chimeric antigen receptors or combinations thereof.

For example, TCR receptors can include various recombinant proteins derived from TCR, comprising extracellular antigen-binding domains (also called antigen recognition units), TCR transmembrane domains, and intracellular domains. They are generally capable of: i) binding to surface antigens on target cells; ii) interacting with other polypeptide components of the complete TCR complex when localized to T cells. In some embodiments, the TCR-T cells are NK cells. In some embodiments, the TCR-T cells are γδ T cells. In one aspect, the antigen-binding domain of a TCR includes antibody fragments. In another aspect, the TCR includes scFv- or sdAb-comprising antibody fragments. In one embodiment, the antigen-binding domain of the TCR comprises an antibody heavy chain variable region and an antibody light chain variable region fused to the constant regions of the TCR subunit α and β chains respectively.

For example, a chimeric antigen receptor (CAR) can include extracellular antigen-binding domains, transmembrane domains, and intracellular signaling domains. Intracellular signaling domains include stimulatory molecules and/or functional signaling domains of costimulatory molecules. In one aspect, the stimulatory molecule is a δ chain that binds to the T cell receptor complex; in one aspect, the cytoplasmic signaling domain further includes functional signaling domains of one or more costimulatory molecules, such as 4-1BB (i.e. CD137), CD27 and/or CD28.

For example, the cells comprise immune cells. For example, the cells comprise immune effector cells. For example, the cells comprise immune effector T cells, immune effector NK cells, and immune effector NKT cells. For example, the cells may comprise phagocytes, lymphocytes, neutrophils, eosinophils, and/or basophils. For example, the cells may comprise monocytes, macrophages, and/or dendritic cells. For example, the cells may comprise B cells, T cells, natural killer cells, and/or natural killer-like T cells. For example, the cells may comprise αβ T cells and/or γδ T cells. For example, the cells may comprise tumor-infiltrating lymphocytes (TILs). For example, the TILs are TILs derived from fragments of tumor tissue, pleural effusion and/or peritoneal effusion, and/or are TILs recovered after cryopreservation. For example, the volume of the fragments ranges from about 1 cubic millimeter to about 27 cubic millimeters.

For example, the cells of the present invention also comprise cells derived from differentiation of stem cells. For example, the cells of the present invention also comprise cells derived from differentiation of pluripotent stem cells. For example, the stem cells of the present invention can be obtained by induction. For example, the above-mentioned stem cells of the present invention may comprise induced pluripotent stem cells (iPSCs), embryonic stem cells, bone marrow stem cells, umbilical cord blood stem cells and/or peripheral blood stem cells.

For example, “stem cells” of the present invention also include pluripotent cells, multipotent cells, precursor cells and progenitor cells. For example, stem cells can be obtained from hematopoietic or mesenchymal stem cells obtained from bone marrow tissue, placental stem cells obtained from placental tissue, embryonic stem cells obtained from embryonic tissue, or embryonic germ cells obtained from reproductive tissue of fetuses. Exemplary pluripotent stem cells can also be generated from somatic cells by reprogramming them to a pluripotent state through the expression of certain transcription factors associated with pluripotency; these cells are called “induced pluripotent stem cells” or “iPSCs”.

For example, the cells comprise B cells, T cells, natural killer cells and/or natural killer-like T cells (NKT). For example, “unmodified cells” or “unengineered cells” may refer to cells or cell populations in which the genome has not been modified and does not contain a gene regulatory system or contains a control gene regulatory system (e.g., empty vector control, non-targeting gRNA, interfering siRNA, etc.). For example, the cells comprise αβ T cells and/or γδ T cells. For example, the cells comprise tumor-infiltrating lymphocytes (TILs). For example, the TILs are TILs derived from tumor tissue, tumor-associated lymph nodes with or without tumor metastasis, metastatic tumor lesions, fragments of peritumoral tissue, pleural effusion and/or peritoneal effusion and/or are TILs recovered after cryopreservation.

For example, the TILs of the present invention may be TILs derived from tumor tissue, tumor-associated lymph nodes with or without tumor metastasis, metastatic tumor lesions, fragments of peritumoral tissue, pleural effusion and/or peritoneal effusion and/or are TILs recovered after cryopreservation. For example, TILs of the present invention can be obtained by processing tumor tissue into tumor fragments. For example, the tumor fragments of the present invention have a volume of approximately 1-27 cubic millimeters. For example, the volume of the tumor fragments of the present invention is about 1 cubic millimeter, about 2 cubic millimeters, about 3 cubic millimeters, about 4 cubic millimeters, about 5 cubic millimeters, about 6 cubic millimeters, about 7 cubic millimeters, about 8 cubic millimeters, about 9 cubic millimeters, about 10 cubic millimeters, about 11 cubic millimeters, about 12 cubic millimeters, about 13 cubic millimeters, about 14 cubic millimeters, about 15 cubic millimeters, about 16 cubic millimeters, about 17 cubic millimeters, about 18 cubic millimeters, about 19 cubic millimeters, about 20 cubic millimeters, about 21 cubic millimeters, about 23 cubic millimeters, about 24 cubic millimeters, about 25 cubic millimeters, about 26 cubic millimeters or about 27 cubic millimeters.

For example, the cells comprise engineered immune receptors displayed on the cell surface. For example, the engineered immune receptor specifically binds to antigens expressed on target cells. For example, the cells comprise chimeric antigen receptors and/or T cell receptors.

The invention provides a type of cell, which is cryopreserved by the method of the invention. For example. During or after thawing of the preserved biological sample, at least a portion of the cells/T cells/engineered T cells, or most of the cells/T cells/engineered T cells in the sample remain viable and/or retain at least part of their biological functions. In one aspect, during or after thawing of the preserved biological sample, at least a certain percentage of the cells in the sample, about or more than about 20%, about 30%, about 40%, about 50%, about 60%, about 70%, about 80%, about 90%, or about 100%, remain viable and/or retain biological function related to their apoptotic markers or indicators. For example, slow freezing conditions can allow intracellular water to exit the cell before freezing, and preservation at low temperatures can reduce the formation of ice crystals.

### Cryopreservation Program Two

In one aspect, the invention provides a cell cryopreservation method, comprising the following steps:
Step 1: Mix a cell sample and a cryopreservation agent to obtain a cryopreservation sample, put the cryopreservation sample into a cryopreservation container, and then put the cryopreservation container into cryopreservation equipment to form a cryopreservation system;
Step 2: Configure the cryopreservation equipment and perform the following steps:
(1) Reduce the overall temperature of the cryopreservation system to approximately 5~15 degrees Celsius.
(2) Reduce the temperature to or maintain at about 1 to 5 degrees Celsius, where the temperature decreases at a rate of about 1 degree Celsius per minute;
(3) Maintain at about 1 to 5 degrees Celsius for about 3 minutes;
(4) Reduce the temperature to about −1~−6 degrees Celsius, where the temperature decreases at a rate of about 0.5~1.5 degrees Celsius per minute;
(5) Reduce the temperature to about −40~−65 degrees Celsius, where the temperature decreases at a rate of about 25~40 degrees Celsius per minute;
(6) Increase the temperature to about −8~−10 degrees Celsius, where the temperature increases at a rate of about 25~35 degrees Celsius per minute;
(7) Reduce the temperature to about −13~−15 degrees Celsius, where the temperature decreases at a rate of about 1.5~3 degrees Celsius per minute;
(8) Maintain at about −8~−15 degrees Celsius for about 5 minutes;
(9) Reduce the temperature to about −30~−40 degrees Celsius, where the temperature decreases at a rate of about 1~3 degrees Celsius per minute;
(10) Reduce the temperature to about −80 to −90 degrees Celsius, where the temperature decreases at a rate of about 10~20 degrees Celsius per minute;
(11) Finally, maintain at about −80 to −90 degrees Celsius for about 10 minutes.

In a preferred embodiment, the cryopreservation equipment is configured to perform the following steps:
(1) Reduce the overall temperature of the cryopreservation system to about 10 degrees Celsius,
(2) Reduce the temperature to about 5 degrees Celsius, where the temperature decreases at a rate of about 1 degree Celsius per minute;
(3) Maintain at about 5 degrees Celsius for about 3 minutes;
(4) Reduce the temperature to about −2 degrees Celsius, where the temperature decreases at a rate of about 0.5 degrees Celsius per minute;
(5) Reduce the temperature to about −60 degrees Celsius, where the temperature decreases at a rate of about 35 degrees Celsius per minute;
(6) Increase the temperature to about −10 degrees Celsius, where the temperature increases at a rate of about 30 degrees Celsius per minute;
(7) Reduce the temperature to about −15 degrees Celsius, where the temperature decreases at a rate of about 3 degrees Celsius per minute;
(8) Maintain at about −15 degrees Celsius for about 5 minutes;
(9) Reduce the temperature to about −35 degrees Celsius, where the temperature decreases at a rate of about 2 degrees Celsius per minute;
(10) Reduce the temperature to about −90 degrees Celsius, where the temperature decreases at a rate of about 15 degrees Celsius per minute;
(11) Finally, maintain at about −90 degrees Celsius for about 10 minutes.

In one aspect, the invention provides a cryopreservation method, comprising the following steps:
(1) Reduce the overall temperature of the cryopreservation system to about 5 to 15 degrees Celsius,
(2) Reduce the temperature to or maintain at about 1 to 5 degrees Celsius, where the temperature decreases at a rate of about 0.5 to 1 degrees Celsius per minute;
(3) Maintain at about 1 to 5 degrees Celsius for about 3 to 5 minutes;
(4) Reduce the temperature to about −1 to −4 degrees Celsius, where the temperature decreases at a rate of about 0.5 to 1.5 degrees Celsius per minute;
(5) Reduce the temperature to about −50 to −60 degrees Celsius, where the temperature decreases at a rate of about 25 to 35 degrees Celsius per minute;
(6) Increase the temperature to about −8 to −10 degrees Celsius, where the temperature increases at a rate of about 25 to 35 degrees Celsius per minute;
(7) Reduce the temperature to about −13 to −15 degrees Celsius, where the temperature decreases at a rate of about 1.5 to 3 degrees Celsius per minute;
(8) Maintain at about −13 to −15 degrees Celsius for about 3 to 5 minutes;
(9) Reduce the temperature to about −35 to −45 degrees Celsius, where the temperature decreases at a rate of about 1 to 2 degrees Celsius per minute;
(10) Reduce the temperature to about −80 to −90 degrees Celsius, where the temperature decreases at a rate of about 10 to 20 degrees Celsius per minute;
(11) Finally, maintain at about −80 to −90 degrees Celsius for at least about 3 minutes.

In one aspect, the invention provides a cryopreservation method, comprising the following steps:
(1) Reduce the overall temperature of the cryopreservation system to about 5 to 15 degrees Celsius,
(2) Reduce the temperature to or maintain at about 3 to 5 degrees Celsius, where the temperature decreases at a rate of about 0.5 to 1 degrees Celsius per minute;
(3) Maintain at about 3 to 5 degrees Celsius for about 3 minutes;
(4) Reduce the temperature to about −1 to −4 degrees Celsius, where the temperature decreases at a rate of about 0.5 to 1.5 degrees Celsius per minute;
(5) Reduce the temperature to about −50 to −60 degrees Celsius, where the temperature decreases at a rate of about 25 to 35 degrees Celsius per minute;
(6) Increase the temperature to about −8 to −10 degrees Celsius, where the temperature increases at a rate of about 25 to 35 degrees Celsius per minute;
(7) Reduce the temperature to about −13 to −15 degrees Celsius, where the temperature decreases at a rate of about 1.5 to 3 degrees Celsius per minute;
(8) Maintain at about −13 to −15 degrees Celsius for about 3 to 5 minutes;
(9) Reduce the temperature to about −35 to −45 degrees Celsius, where the temperature decreases at a rate of about 2 degrees Celsius per minute;
(10) Reduce the temperature to about −90 degrees Celsius, where the temperature decreases at a rate of about 10 to 20 degrees Celsius per minute;
(11) Finally, maintain at about −90 degrees Celsius for about 10 minutes.

Among these, the cryopreservation system usually refers to placing the materials to be cryopreserved in a controlled cooling environment to form a system. For example, the controlled cooling environment can be a cooling compartment of a cooling equipment, a cooling chamber, etc. For example, the method provided by the present invention uses steps to cause the ambient temperature of the cryopreservation system to increase or decrease at a set rate, or be maintained at a set temperature, so that the material to be cryopreserved can cool down gradually and pass through the crystallization nucleation point smoothly, avoiding damage to cells caused by intracellular crystallization. For example, the method provided by the present invention uses steps to cause the temperature in the chamber of the cooling instrument to increase or decrease at a set rate, or be maintained at a set temperature.

For example, the cooling control program of the method of the present invention can be to control the cooling rate and the cooling end point of the temperature in the cooling instrument chamber, or can be to control the maintaining temperature and maintaining time of the temperature in the cooling instrument chamber.

In some embodiments, in step one of the method, a cell sample and a cryopreservation agent are mixed in a volume ratio of 1:0.5~1:10, wherein the cryopreservation agent comprises: 10~50% (w/v) of CS 10 or DMSO, 2~25% (w/v) of 20% human albumin injection. For example, the cryopreservation agent may comprise CS10. For example, the cryopreservation again may comprise DMSO. For example, the cryopreservation agent comprises (w/v): 10%~50% of DMSO, for example, about 10%, about 15%, about 20%, about 25%, about 30%, about 35%, about 40%, about 45%, about 50% (w/v) of DMSO; its concentration in the cryopreservation sample is about 2.0% to 6.0%, or about 2.0% to 4.5%, or about 2.0% to 4.0%, or about 2.0% to 3.8%, or about 2.0% to 3.75%, or about 2.0% to 3.0%, or about 2.5% to 4.5%, or about 2.5% to 4.0%, or about 2.5% to 3.8%, or about 2.5% to 3.75%, or about 2.5% to 3.0%, or about 3.0% to 4.5%, or about 3.0% to 4.0%, or about 3.0% to 3.8%, or about 3.0% to 3.75%, or about 3.75% To 4.5%, or about 3.8% to 4.5%, or about 3.75% to 4.0%, or about 3.8% to 4.0%, or about 4.0% to 4.5% (w/v). For example, the cryopreservation agent can comprise (w/v): 10~50% of CS 10. For example, the cryopreservation agent can comprise about 10%, about 15%, about 20%, about 25%, about 30%., about 35%, about 40%, about 45%, about 50% (w/v) of CS 10, with a concentration in the cryopreservation sample of about 2.0% to 6.0%, or about 2.0% to 4.5%, or about 2.0% to 4.0%, or about 2.0% to 3.8%, or about 2.0% to 3.75%, or about 2.0% to 3.0%, or about 2.5% to 4.5%, or about 2.5% to 4.0%, or about 2.5% to 3.8%, or about 2.5% to 3.75%, or about 2.5% to 3.0%, or about 3.0% to 4.5%, or about 3.0% to 4.0%, or about 3.0% to 3.8%, or about 3.0% to 3.75%, or about 3.75% to 4.5%, or about 3.8% to 4.5%, or about 3.75% to 4.0%, or about 3.8% to 4.0%, or about 4.0% to 4.5% (w/v). For example, the cryopreservation agent may also comprise human albumin. For example, the cryopreservation agent may comprise about 2%, about 3%, about 5%, about 10%, about 15%, about 20%, about 25% (w/v) of 20% human albumin injection. In some embodiments, the cryopreservation agent also comprises an electrolyte injection solution. For example, the cryopreservation agent comprises 25%~88% (w/v) compound electrolyte injection solution. The above percentages are based on the total volume of the cryopreservation agent.

In some embodiments, in step one of the method, the cell sample is prepared by mixing cells and culture medium at a volume ratio of 1:0.1~1:50, preferably 1:0.5~1:10. The culture medium may comprise commercial cell culture media, such as basal culture media (1640 or DMEM) known in the art. For example, the culture medium of the present invention may comprise serum, or be serum-free. For example, the cells and culture medium are mixed at a volume ratio of about 1:0.1, about 1:0.2, about 1:0.5, about 1:1, about 1:2, about 1:5, about 1:10, about 1:20, or about 1:50. For example, the medium comprises 5~50% serum (e.g., FBS) and 10~90% basal culture medium, based on the total volume of the medium. For example, cells can be suspended in cell culture medium at the following density: approximately 1×10⁵ cells/mL to approximately 2×10⁸ cells/mL, about 1×10⁵ cells/mL to approximately 1×10⁸ cells/mL, about 1×10⁶ cells/mL to approximately 1×10⁸ cells/mL, about 1×10⁷ cells/mL to approximately 1×10⁸ cells/mL, or about 4×10⁷ cells/mL to 1×10⁸ cells/mL. In some embodiments, cells are suspended in the cell culture medium at the following density: about 1×10⁵ cells/mL, about 5×10⁵ cells/mL, about 1×10⁶ cells/mL, about 2×10⁶ cells/mL, about 5×10⁶ cells/mL, about 1×10⁷ cells/mL, about 1.5×10⁷ cells/mL, about 2×10⁷ cells/mL, about 2.5×10⁷ cells/mL, about 3×10⁷ cells/mL, about 3.5×10⁷ cells/mL, about 4×10⁷ cells/mL, about 4.5×10⁷ cells/mL, about 5.0×10⁷ cells/mL, about 5.5×10⁷ cells/mL, about 6.0×10⁷ cells/mL, about 6.5×10⁷ cells/mL, about 7.0×10⁷ cells/mL, about 7.5×10⁷ cells/mL, about 8.0×10⁷ cells/mL, about 8.5×10⁷ cells/mL, about 9.0×10⁷ cells/mL, about 9.5×10⁷ cells/mL, about 1×10⁸ cells/mL, about 1.5×10⁸ cells/mL or approximately 2×10⁸ cells/mL. In some embodiments, cells are suspended at a density of approximately 1.5×10⁷ cells/mL to approximately 6×10⁷ cells/mL in the cell culture medium. In some embodiments, cells are suspended at a density of approximately 5×10⁶ cells/mL to approximately 150×10⁶ cells/mL in the cell culture medium. In some embodiments, cells are suspended at a density of at least about 1×10⁷ cells/mL in the cell culture medium. In a certain embodiment, cells are suspended at a density of at least about 5.0×10⁷ cells/mL in the cell culture medium. In some embodiments, the cells are viable cells. In some embodiments, cell density is calculated and determined by measuring cell diameter. In some embodiments, cell density is determined by a cell counting instrument.

In some embodiments, the method may also comprise storing the cryopreservation container in an environment of about −80 degrees Celsius or below after the cooling of the cryopreservation system is completed (that is, after completing the above step (11)). For example, the method may also comprise storing the cryopreservation container in liquid nitrogen after the cooling of the cryopreservation system is completed. For example, the maintained ambient temperature may be at a temperature from −196° C. to −80° C.

In some embodiments, the cryopreservation container may comprise cryopreservation bags and/or cryovials.

In some embodiments, the volume of the cryopreservation sample in the cryopreservation container is about 1 microliter to 1 liter. For example, the volume of the cryopreservation sample in the cryopreservation container can be about 1 microliter, about 10 microliters, about 100 microliters, about 1000 microliters, about 1.5 milliliters, about 2 milliliters, about 2.5 milliliters, about 3.5 milliliters, about 4 milliliters, about 5 milliliters, about 10 milliliters, about 20 milliliters, about 25 milliliters, about 50 milliliters, about 75 milliliters, about 100 milliliters, about 150 milliliters, about 200 milliliters, about 250 milliliters, about 500 milliliters, about 750 milliliters, or about 1000 milliliters. For example, a 2 ml cryovial can be used to hold 1 ml of the cryopreservation sample, a 50 ml cryopreservation bag can be used to hold 10 ml of the cryopreservation sample, or a 750 ml cryopreservation bag can be used to hold 90 ml of the cryopreservation sample.

In some embodiments, the density of cells in the cryopreservation sample is approximately 1×10⁵ to 1×10⁹ cells/mL.

In some embodiments, the cells can comprise immune cells.

For example, immune cells enriched in samples of the present invention include CD4⁺ T cells or subsets thereof and/or CD8⁺ T cells or subsets thereof. For example, CD4⁺ cell subsets and/or CD8⁺ cell subsets are optionally selected from the following cells and combinations thereof: memory cells, central memory T (T_{CM}) cells, effector memory cells (T_{EM}), stem cell-like central memory (T_{SCM}) cells, T effector (T_{E}) cells, naive T (T_{N}) cells, regulatory T (T_{REG}) cells and/or helper T cells; or TH1 cells, TH2 cells, TH3 cells, TH17 cells, TH9 cells, TH22 cells, follicular helper T cells, α/β T cells and δ/γ T cells. For example, samples of the invention comprise primary T cells obtained from a subject. For example, samples of the invention include one or more subsets of T cells or other types of cells, such as the entire T cell population, CD4⁺ cells, CD8⁺ cells and their subsets. For example, samples of the invention include T cells isolated from a PBMC sample by negative selection for markers expressed on non-T cells, such as B cells, monocytes or other leukocytes, such as CD14. In some aspects, CD4⁺ or CD8⁺ selection steps are used to isolate CD4⁺ helper T cells and CD8⁺ cytotoxic T cells. CD4⁺ and CD8⁺ populations may be further grouped into subsets by positive or negative selection for markers expressed on or to a relatively higher degree on one or more naive T cell, memory T cell and/or effector T cell subsets. For example, biological samples include T cells and/or engineered T cells. For example, engineered T cells include T cells that express recombinant molecules or exogenous molecules, which are optionally recombinant proteins, optionally recombinant receptors, which are optionally T cell receptors (TCR), chimeric receptors, chimeric antigen receptors or combinations thereof.

For example, TCR receptors can include various recombinant proteins derived from TCR, comprising extracellular antigen-binding domains (also called antigen recognition units), TCR transmembrane domains, and intracellular domains. They are generally capable of: i) binding to surface antigens on target cells; ii) interacting with other polypeptide components of the complete TCR complex when localized to T cells. In some embodiments, the cells expressing the TCR are NK cells. In some embodiments, the cells expressing the TCR are NKT cells. In some embodiments, the TCR-T cells are γδ T cells. In one aspect, the antigen-binding domain of a TCR includes antibody fragments. In another aspect, the TCR includes scFv- or sdAb-comprising antibody fragments. In one example, the antigen-binding domain of the TCR comprises an antibody heavy chain variable region and an antibody light chain variable region fused to the constant regions of the TCR subunit α and β chains respectively.

For example, a chimeric antigen receptor (CAR) can include extracellular antigen-binding domains, transmembrane domains, and intracellular signaling domains. Intracellular signaling domains include stimulatory molecules and/or functional signaling domains of costimulatory molecules. In one aspect, the stimulatory molecule is a δ chain that binds to the T cell receptor complex; in one aspect, the cytoplasmic signaling domain further includes functional signaling domains of one or more costimulatory molecules, such as 4-1BB (i.e. CD 137), CD 27 and/or CD28.

For example, the cells comprise immune cells. For example, the cells comprise immune effector cells. For example, the cells comprise immune effector T cells, immune effector NK cells, and immune effector NKT cells. For example, the cells comprise phagocytes, lymphocytes, neutrophils, eosinophils and/or basophils.

For example, the cells comprise monocytes, macrophages, and/or dendritic cells.

For example, the cells of the present invention also comprise cells derived from differentiation of stem cells. For example, the cells of the present invention also comprise cells derived from differentiation of pluripotent stem cells. For example, the stem cells of the present invention can be obtained by induction. For example, the above-mentioned stem cells of the present invention may comprise induced pluripotent stem cells (iPSCs), embryonic stem cells, bone marrow stem cells, umbilical cord blood stem cells and/or peripheral blood stem cells.

For example, “stem cells” of the present invention also include pluripotent cells, multipotent cells, precursor cells and progenitor cells. For example, stem cells can be obtained from hematopoietic or mesenchymal stem cells obtained from bone marrow tissue, placental stem cells obtained from placental tissue, embryonic stem cells obtained from embryonic tissue, or embryonic germ cells obtained from reproductive tissue of the fetuses. Exemplary pluripotent stem cells can also be generated from somatic cells by reprogramming them to a pluripotent state through the expression of certain transcription factors associated with pluripotency; these cells are called “induced pluripotent stem cells” or “iPSCs”.

For example, the cells comprise B cells, T cells, natural killer cells and/or natural killer-like T cells (NKT). For example, “unmodified cells” or “unengineered cells” may refer to cells or cell populations in which the genome has not been modified and does not contain a gene regulatory system or contains a control gene regulatory system (e.g., empty vector control, non-targeting gRNA, interfering siRNA, etc.). For example, the cells comprise αβ T cells and/or γδ T cells. For example, the cells comprise tumor-infiltrating lymphocytes (TILs). For example, the TILs are TILs derived from tumor tissue, tumor-associated lymph nodes with or without tumor metastasis, metastatic tumor lesions, fragments of peritumoral tissue, pleural effusion and/or peritoneal effusion and/or are TILs recovered after cryopreservation.

For example, the TILs of the present invention may be TILs derived from tumor tissue, tumor-associated lymph nodes with or without tumor metastasis, metastatic tumor lesions, fragments of peritumoral tissue, pleural effusion and/or peritoneal effusion and/or are TILs recovered after cryopreservation. For example, TILs of the present invention can be obtained by processing tumor tissue into tumor fragments. For example, the tumor fragments of the present invention have a volume of approximately 1-27 cubic millimeters. For example, the volume of the tumor fragments of the present invention is about 1 cubic millimeter, about 2 cubic millimeters, about 3 cubic millimeters, about 4 cubic millimeters, about 5 cubic millimeters, about 6 cubic millimeters, about 7 cubic millimeters, about 8 cubic millimeters, about 9 cubic millimeters, about 10 cubic millimeters, about 11 cubic millimeters, about 12 cubic millimeters, about 13 cubic millimeters, about 14 cubic millimeters, about 15 cubic millimeters, about 16 cubic millimeters, about 17 cubic millimeters, about 18 cubic millimeters, about 19 cubic millimeters, about 20 cubic millimeters, about 21 cubic millimeters, about 23 cubic millimeters, about 24 cubic millimeters, about 25 cubic millimeters, about 26 cubic millimeters or about 27 cubic millimeters.

For example, the cells comprise engineered immune receptors displayed on the cell surface. For example, the engineered immune receptor specifically binds to antigens expressed on target cells. For example, the cells comprise chimeric antigen receptors and/or T cell receptors.

In another aspect, the present invention provides a cell cryopreservation system, which comprises cryopreservation equipment configured to perform the following cryopreservation program:
(1) Reduce the overall temperature of the cryopreservation system to approximately 5~15 degrees Celsius.
(2) Reduce the temperature to about 1 to 5 degrees Celsius, where the temperature decreases at a rate of about 1 degree Celsius per minute;
(3) Maintain at about 5 degrees Celsius for about 3 minutes;
(4) Reduce the temperature to about −2~−6 degrees Celsius, where the temperature decreases at a rate of about 0.5~1.5 degrees Celsius per minute;
(5) Reduce the temperature to about −40~−65 degrees Celsius, where the temperature decreases at a rate of about 25~40 degrees Celsius per minute;
(6) Increase the temperature to about −8~−10 degrees Celsius, where the temperature increases at a rate of about 25~35 degrees Celsius per minute;
(7) Reduce the temperature to about −13~−15 degrees Celsius, where the temperature decreases at a rate of about 1.5~3 degrees Celsius per minute;
(8) Maintain at about −8~−15 degrees Celsius for about 5 minutes;
(9) Reduce the temperature to about −30~−40 degrees Celsius, where the temperature decreases at a rate of about 1~3 degrees Celsius per minute;
(10) Reduce the temperature to about −90 degrees Celsius, where the temperature decreases at a rate of about 10~20 degrees Celsius per minute;
(11) Finally, maintain at about −90 degrees Celsius for about 10 minutes.

In a preferred embodiment, the cryopreservation equipment is configured to perform the following cryopreservation program:
(1) Reduce the overall temperature of the cryopreservation system to about 10 degrees Celsius,
(2) Reduce the temperature to about 5 degrees Celsius, where the temperature decreases at a rate of about 1 degree Celsius per minute;
(3) Maintain at about 5 degrees Celsius for about 3 minutes;
(4) Reduce the temperature to about −2 degrees Celsius, where the temperature decreases at a rate of about 0.5 degrees Celsius per minute;
(5) Reduce the temperature to about −60 degrees Celsius, where the temperature decreases at a rate of about 35 degrees Celsius per minute;
(6) Increase the temperature to about −10 degrees Celsius, where the temperature increases at a rate of about 30 degrees Celsius per minute;
(7) Reduce the temperature to about −15 degrees Celsius, where the temperature decreases at a rate of about 3 degrees Celsius per minute;
(8) Maintain at about −15 degrees Celsius for about 5 minutes;
(9) Reduce the temperature to about −35 degrees Celsius, where the temperature decreases at a rate of about 2 degrees Celsius per minute;
(10) Reduce the temperature to about −90 degrees Celsius, where the temperature decreases at a rate of about 15 degrees Celsius per minute;
(11) Finally, maintain at about −90 degrees Celsius for about 10 minutes.

### TIL Cell Culture

The present invention provides a method for obtaining tumor infiltrating lymphocytes (TIL). The method of obtaining TIL cells from subject tissue samples can be to obtain primary tumor samples or metastatic tumor samples from the patient during surgery, and the weight can be at least about 1 g, or multiple pieces of tissue can be combined. The tumor tissue, pleural effusion and/or peritoneal effusion are transported in a sample transport solution, such as a commercially available tumor tissue transport solution, tumor tissue preservation solution or tumor tissue transfer solution at about 2-8 degrees Celsius, and processed within 48 hours. The tissue pieces can be mechanically fragmented to a size of about 1-27 cubic millimeters each, transferred into a gas permeable culture bag or Grex, and added with cell serum-free medium and IL-2 at a concentration of 300-9000 IU/mL (for example, 1000-9000 IU/mL, for example 6000 IU/mL) for about 3-14 days, and optionally IL-7 and/or IL-15 can also be added. The cells in the culture medium are collected, transferred to a gas permeable culture bag, or Grex, or Xuri equipment, where the cell serum-free culture medium can be supplemented with CD28 antibodies, CD3 antibodies and CD28 antibodies of the present invention, magnetic beads comprising CD3 antibodies and CD28 antibodies (such as Dynabeads) , and/or a nanomatrix (such as transACT) containing CD3 antibodies and CD28 antibodies, IL-2 at a concentration of 300-9000 IU/mL (for example 1000-9000 IU/mL, for example 6000 IU/mL), and optionally IL-7 and/or IL-15, and, after activating the TILs of the present invention for a certain period of time, supplemented with irradiated PBMCs (the ratio of TILs to PBMCs is about 1:40 to about 1:400), and expanded for about 3-14 days. Cell processing systems can be used for collecting cells in the culture medium, washing and cryopreservation, and detection. The CD3 ratio of the final product can be greater than 80%, the cell viability can be greater than 50%, and greater than 80% of the cells can be memory effector cells and effector cells. After stimulation, IFNγ can be secreted, and/or there can be an increase in the proportion of activated cells.

In another aspect, a type of cell is provided, which cell is cryopreserved by the method of the present invention. For example, during or after thawing the preserved cell sample, at least a portion of the cells/T cells/engineered T cells, or most of the cells/T cells/engineered T cells in the sample remain viable and/or retain at least part of their biological functions. In one aspect, during or after thawing the preserved cell sample, at least a certain percentage of the cells in the sample, about or more than about 20%, about 30%, about 40%, about 50%, about 60%, about 70%, about 80%, about 90%, or about 100%, remain viable and/or retain biological function related to their apoptotic markers or indicators. Using the method of the present invention, slow freezing conditions can allow intracellular water to exit the cell before freezing, and preservation at low temperatures can reduce the formation of ice crystals.

In one aspect, the present invention provides a type of cell, and the cell of the present invention can be obtained from the cryopreservation method of the present invention. In one embodiment, the cells provided by the present invention may comprise one type of or one batch of cells preserved by the cryopreservation method of the present invention. In one embodiment, the cells provided by the present invention may comprise multiple types of or multiple batches of cells preserved by the cryopreservation method of the present invention and combined in any proportion.

In some embodiments, cells recovered and cultured after cryopreservation using the method of the present invention can be administered to patients as a pharmaceutical composition. In some embodiments, the pharmaceutical composition can be a suspension of cells in a sterile buffer. For example, cells expanded from PBMCs of the invention may be administered by any suitable route known in the art. In some embodiments, cells can be administered in a single intra-arterial or intravenous infusion, which can last about 30 to 60 minutes. Other suitable routes of administration may include intraperitoneal, intrathecal and intralymphatic administration.

In some embodiments, any suitable dose of cells may be administered. In some embodiments, for example when the tumor is melanoma, at least about 1×10⁹ cells, or about 2.3×10⁹ to about 13.7×10¹⁰ cells may be administered. In some embodiments, about 1×10⁹ to about 12×10¹⁰ cells may be administered. In some embodiments, about 1.2×10¹⁰ to about 4.3×10¹⁰ cells may be administered. In some embodiments, about 3×10¹⁰ to about 12×10¹⁰ cells may be administered. In some embodiments, about 4×10¹⁰ to about 10×10¹⁰ cells may be administered. In some embodiments, about 5×10¹⁰ to about 8×10¹⁰ cells may be administered. In some embodiments, about 6×10¹⁰ to about 8×10¹⁰ cells may be administered. In some embodiments, about 7×10¹⁰ to about 8×10¹⁰ cells may be administered. In some embodiments, the therapeutically effective dose may be from about 2.3×10⁹ to about 13.7×10¹⁰ cells. In some embodiments, the therapeutically effective dose may be from about 1×10⁹ to about 12×10¹⁰ cells. In some embodiments, the therapeutically effective dose may be from about 1.2×10¹⁰ to about 4.3×10¹⁰ cells. In some embodiments, the therapeutically effective dose may be from about 3×10¹⁰ to about 12×10¹⁰ cells. In some embodiments, the therapeutically effective dose may be from about 4×10¹⁰ to about 10×10¹⁰ cells. In some embodiments, the therapeutically effective dose may be from about 5×10¹⁰ to about 8×10¹⁰ cells. In some embodiments, the therapeutically effective dose may be from about 6×10¹⁰ to about 8×10¹⁰ cells. In some embodiments, the therapeutic effective dose may be from about 7×10¹⁰ to about 8×10¹⁰ cells.

In some embodiments, cells can be administered in a single dose. Such administration may be by injection, for example intravenously. In some embodiments, cells can be administered in multiple doses. The dose may be one, two, three, four, five, six, or more than six times per year. The dose can be once monthly, once every two weeks, once a week, or once every 2 days. In some embodiments, administration of cells can be continuous.

In some embodiments, the number of cells provided in the composition of the invention can be about 1×10⁶ to 9×10¹³, for example about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹, about 2×10⁹, about 3×10⁹, about 4×10⁹, about 5×10⁹, about 6×10⁹, about 7×10⁹, about 8×10⁹, about 9×10⁹, about 1×10¹⁰, about 2×10¹⁰, about 3×10¹⁰, about 4×10¹⁰, about 5×10¹⁰, about 6×10¹⁰, about 7×10¹⁰, about 8×10¹⁰ about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹, about 1×10¹², about 2×10¹², about 3×10¹² about 4×10¹², about 5×10¹², about 6×10¹², about 7×10¹², about 8×10¹², about 9×10¹², about 1×10¹³, about 2×10¹³, about 3×10¹³, about 4×10¹³, about 5×10¹³, about 6×10¹³, about 7×10¹³, about 8×10¹³, or about 9×10¹³. In some embodiments, the range of the number of cells provided in the composition of the invention may be from about 1×10⁶ to 5×10⁶, from about 5×10⁶ to 1×10⁷, from about 1×10⁷ to 5×10⁷, about 5×10⁷ to 1×10⁸, about 1×10⁸ to 5 108, about 5×10⁸ to 1×10⁹, about 1×10⁹ to 5×10⁹, approximately 5×10⁹ to 1×10¹⁰, approximately 1×10¹⁰ to 5×10¹⁰ approximately 5×10¹⁰ to 1×10¹¹, approximately 1×10¹¹ to 5×10¹¹, approximately 5×10¹¹ to 1×10¹², approximately 1×10¹² to 5×10¹², approximately 5×10¹² to 1×10¹³, approximately 1×10¹³ to 5×10¹³, or approximately 5×10¹³ to 9×10¹³.

In some embodiments, the concentration of cells provided in the compositions of the invention may be less than about 100% to about 0.0001% of the composition, for example about 100%, about 90%, about 80%, about 70%, about 60%, about 50%, about 40%, about 30%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001% w/w, w/v or v/v.

In some embodiments, the concentration of cells provided in the composition of the invention can be greater than about 90% to about 0.0001% of the composition, for example about 90%, about 80%, about 70%, about 60%, about 50%, about 40%, about 30%, about 20%, about 19.75%, about 19.50%, about 19.25%, about 19%, about 18.75%, about 18.50%, about 18.25%, about 18%, about 17.75%, about 17.50%, about 17.25%, about 17%, about 16.75%, about 16.50%, about 16.25%, about 16%, about 15.75%, about 15.50%, about 15.25%, about 15%, about 14.75%, about 14.50%, about 14.25%, about 14%, about 13.75%, about 13.50%, about 13.25%, about 13%, about 12.75%, about 12.50%, about 12.25%, about 12%, about 11.75%, about 11.50%, about 11.25%, about 11%, about 10.75%, about 10.50%, about 10.25%, about 10%, about 9.75%, about 9.50%, about 9.25%, about 9%, about 8.75%, about 8.50%, about 8.25%, about 8%, about 7.75%, about 7.50%, about 7.25%, about 7%, about 6.75%, about 6.50%, about 6.25%, about 6%, about 5.75%, about 5.50%, about 5.25%, about 5%, about 4.75%, about 4.50%, about 4.25%, about 4%, about 3.75%, about 3.50%, about 3.25%, about 3%, about 2.75%, about 2.50%, about 2.25%, about 2%, about 1.75%, about 1.50%, about 1.25%, about 1%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about or 0.0002%, or about 0.0001% w/w, w/v or v/v.

In some embodiments, the concentration of cells provided in the compositions of the invention may range from about 0.0001% to about 50%, from about 0.001% to about 40%, from about 0.01% to about 30%, about 0.02% of the composition. to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, or about 1% to about 10% w/w, w/v or v/v.

In some embodiments, the concentration of cells provided in the composition of the invention may range from about 0.001% to about 10%, from about 0.01% to about 5%, from about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, or about 0.1% to about 0.9% of the composition w/w, w/v or v/v.

In some embodiments, the amount of cells provided in the compositions of the invention can be equal to or less than about 10 g, about 9.5 g, about 9.0 g, about 8.5 g, about 8.0 g, about 7.5 g, about 7.0 g, about 6.5 g, about 6.0 g, about 5.5 g, about 5.0 g, about 4.5 g, about 4.0 g, about 3.5 g, about 3.0 g, about 2.5 g, about 2.0 g, about 1.5 g, about 1.0 g, about 0.95 g, About 0.9 g, about 0.85 g, about 0.8 g, about 0.75 g, about 0.7 g, about 0.65 g, about 0.6 g, about 0.55 g, about 0.5 g, about 0.45 g, about 0.4 g, about 0.35 g, about 0.3 g, about 0.25 g, about 0.2 g, about 0.15 g, about 0.1 g, about 0.09 g, about 0.08 g, about 0.07 g, about 0.06 g, about 0.05 g, about 0.04 g, about 0.03 g, about 0.02 g, about 0.01 g, about 0.009 g, about 0.008 g, about 0.007 g, about 0.006 g, about 0.005 g, about 0.004 g, about 0.003 g, about 0.002 g, about 0.001 g, about 0.0009 g, about 0.0008 g, about 0.0007 g, about 0.0006 g, about 0.0005 g, about 0.0004 g, about 0.0003 g, about 0.0002 g, or about 0.0001 g.

In some embodiments, the amount of cells provided in the compositions of the invention can be greater than about 0.0001 g, about 0.0002 g, about 0.0003 g, about 0.0004 g, about 0.0005 g, about 0.0006 g, about 0.0007 g, about 0.0008 g, about 0.0009 g, about 0.001 g, about 0.0015 g, about 0.002 g, about 0.0025 g, about 0.003 g, about 0.0035 g, about 0.004 g, about 0.0045 g, about 0.005 g, about 0.0055 g, about 0.006 g, about 0.0065 g, about 0.007 g, about 0.0075 g, about 0.008 g, about 0.0085 g, about 0.009 g, about 0.0095 g, about 0.01 g, about 0.015 g, about 0.02 g, about 0.025 g, about 0.03 g, about 0.035 g, about 0.04 g, about 0.045 g, about 0.05 g, about 0.055 g, about 0.06 g, about 0.065 g, about 0.07 g, about 0.075 g, about 0.08 g, about 0.085 g, about 0.09 g, about 0.095 g, about 0.1 g, about 0.15 g, about 0.2 g, about 0.25 g, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, or about 10 g.

In another aspect, the present invention provides a pharmaceutical composition, which may comprise the cells of the present invention and/or the composition of the present invention, and pharmaceutically acceptable carriers.

In another aspect, the present invention provides a kit, which may comprise the cells of the present invention and/or the pharmaceutical composition of the present invention.

In another aspect, the present invention provides a kit. The kit of the present invention may comprise cell activators, cell growth factors and/or feeder cells for the cell culture method of the present invention and instructions describing the steps of the cell culture method of the present invention.

In another aspect, the invention provides a method of affecting the growth of cells (e.g., tumor cells), which may comprise administering to a subject the cells of the invention and/or the pharmaceutical composition of the invention. In some embodiments, affecting tumor growth may comprise reducing the volume of the tumor to about 99% to about 0.1% of that pre-administration, for example about 99%, about 95%, about 90%, about 80%, about 70%, about 60% %, about 50%, about 40%, about 30%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.5%, about 0.4%, about 0.3%, about 0.2%, or about 0.1%.

In another aspect, the present invention provides a use of the cells of the present invention and/or the pharmaceutical composition of the present invention in the preparation of medicaments, and the medicaments of the present invention can be used to prevent and/or treat diseases and/or symptoms. For example, diseases and/or symptoms of the present invention may comprise tumors. In some embodiments, the tumor of the invention is selected from solid tumors. In some embodiments, the tumor of the invention can be one or more types selected from the following group: melanoma, ovarian cancer, cervical cancer, lung cancer, bladder cancer, breast cancer, head and neck cancer, pancreatic cancer, liver cancer, gastric cancer, colorectal cancer, and kidney cancer.

In another aspect, the present invention provides a method for preventing and/or treating diseases and/or symptoms, which may comprise administering the cells of the present invention and/or the pharmaceutical composition of the present invention to a subject. For example, diseases and/or symptoms of the present invention may comprise tumors. In some embodiments, the tumor of the invention is selected from solid tumors. In some embodiments, the tumor of the invention can be one or more types selected from the following group: melanoma, ovarian cancer, cervical cancer, lung cancer, bladder cancer, breast cancer, head and neck cancer, pancreatic cancer, liver cancer, gastric cancer, colorectal cancer, and kidney cancer.

The present invention also includes the following technical solutions:

A cell cryopreservation method comprising the following steps:
Step 1: Mix a cell sample and a cryopreservation agent to obtain a cryopreservation sample, put the cryopreservation sample into a cryopreservation container, and then put the cryopreservation container into cryopreservation equipment to form a cryopreservation system;
Step 2: Configure the cryopreservation equipment and perform the following steps:
(1) Reduce the overall temperature of the cryopreservation system to approximately 5~15 degrees Celsius.
(2) Reduce the temperature to or maintain at about 1 to 5 degrees Celsius, where the temperature decreases at a rate of about 1 degree Celsius per minute;
(3) Maintain at about 1 to 5 degrees Celsius for about 3 minutes;
(4) Reduce the temperature to about −1~−6 degrees Celsius, where the temperature decreases at a rate of about 0.5~1.5 degrees Celsius per minute;
(5) Reduce the temperature to about −40~−65 degrees Celsius, where the temperature decreases at a rate of about 25~40 degrees Celsius per minute;
(6) Increase the temperature to about −8~−10 degrees Celsius, where the temperature increases at a rate of about 25~35 degrees Celsius per minute;
(7) Reduce the temperature to about −13~−15 degrees Celsius, where the temperature decreases at a rate of about 1.5~3 degrees Celsius per minute;
(8) Maintain at about −8~−15 degrees Celsius for about 5 minutes;
(9) Reduce the temperature to about −30~−40 degrees Celsius, where the temperature decreases at a rate of about 1~3 degrees Celsius per minute;
(10) Reduce the temperature to about −80~−90 degrees Celsius, where the temperature decreases at a rate of about 10~20 degrees Celsius per minute;
(11) Finally, maintain at about −80~−90 degrees Celsius for about 10 minutes.

The method of technical solution 1, wherein in step 1, the cell sample and the cryopreservation agent are mixed in a volume ratio of 1:0.5~1:10.

The method of technical solution 2, wherein the cryopreservation agent comprises: 10~50% (w/v) of CS 10 or DMSO, 2~25% (w/v) of 20% human albumin injection, based on the total volume of the cryopreservation agent.

The method of any one of technical solutions 1-3, wherein the cell sample is a mixture of cells and culture medium at a volume ratio of 1:0.5~1:10.

The method of technical solution 4, wherein the culture medium comprises: 5~50% of serum, 10%~90% of basal medium, based on the total volume of the medium; wherein the basal medium is, for example, 1640 or DMEM.

The method of any one of technical solutions 1-5, wherein the method further comprises, after the cooling of the cryopreservation system in step (11) is completed, storing the cryopreservation container in an environment of about −80 degrees Celsius or below.

The method of any one of technical solutions 1-5, wherein the method further comprises storing the cryopreservation container in liquid nitrogen after the cooling of the cryopreservation system in step (11) is completed.

The method of any one of technical solutions 1-7, wherein the cryopreservation container includes cryopreservation bags and/or cryovials.

The method of any one of technical solutions 1-7, wherein the volume of the cryopreserved sample in the cryopreservation container is about 100 microliters to 1 liter.

The method of technical solution 1, wherein the density of cells in the cryopreservation sample is about 1×10⁵ to 1×10⁹ cells/mL.

The method of technical solution 1, wherein the cells comprise immune cells.

The method of technical solution 11, wherein the cells comprise phagocytes, lymphocytes, neutrophils, eosinophils and/or basophils.

The method of technical solution 1, wherein the cells comprise tumor-infiltrating lymphocytes (TILs).

The method of technical solution 13, wherein the TILs are TILs derived from fragments of tumor tissue, pleural effusion and/or peritoneal effusion, and/or are TILs recovered after cryopreservation.

A cell cryopreservation system, which system comprises cryopreservation equipment configured to perform the following cryopreservation program:
(1) Reduce the overall temperature of the cryopreservation system to approximately 5~15 degrees Celsius.
(2) Reduce the temperature to or maintain at about 1 to 5 degrees Celsius, where the temperature decreases at a rate of about 1 degree Celsius per minute;
(3) Maintain at about 1 to 5 degrees Celsius for about 3 minutes;
(4) Reduce the temperature to about −1~−6 degrees Celsius, where the temperature decreases at a rate of about 0.5~1.5 degrees Celsius per minute;
(5) Reduce the temperature to about −40~−65 degrees Celsius, where the temperature decreases at a rate of about 25~40 degrees Celsius per minute;
(6) Increase the temperature to about −8~−10 degrees Celsius, where the temperature increases at a rate of about 25~35 degrees Celsius per minute;
(7) Reduce the temperature to about −13~−15 degrees Celsius, where the temperature decreases at a rate of about 1.5~3 degrees Celsius per minute;
(8) Maintain at about −8~−15 degrees Celsius for about 5 minutes;
(9) Reduce the temperature to about −30~−40 degrees Celsius, where the temperature decreases at a rate of about 1~3 degrees Celsius per minute;
(10) Reduce the temperature to about −80 to −90 degrees Celsius, where the temperature decreases at a rate of about 10~20 degrees Celsius per minute;
(11) Finally, keep it at about −80 to −90 degrees Celsius for about 10 minutes.

The present invention will be further described below in conjunction with specific examples. Without intending to be bound by any theory, the following examples are only for illustrating the methods and uses of the present invention, but are not intended to limit the scope of the present invention. Experimental methods without specifying specific conditions in the following examples usually follow conventional conditions or conditions recommended by the manufacturer. Unless otherwise stated, percentages and parts are by weight.

### EXAMPLES

### Example 1

### Cell Cryopreservation

The cryovial program recommended by the cryopreservation equipment employed (Thermo Scientific) (hereinafter referred to as the “original program”) and the cryopreservation program provided by the present invention (hereinafter referred to as the “new program”) were used for the cryopreservation of cells in the cryovials of the present invention. The main components of the cryopreservation solution within the cryovials were: a DMSO-comprising ready-to-use cryopreservation solution (10~50%), a 20% HAS injection (preparation method: the mass concentration of human serum albumin (HAS) in the injection was 20%) (2~25%), and a compound electrolyte injection (for example, each 1000 mL comprised: sodium chloride 5.26 g; sodium gluconate 5.02 g; sodium acetate 3.68 g; potassium chloride 0.37 g; magnesium chloride 0.30 g) (25~88%).

The cooling process of cryopreservation program 1 of the present invention was as follows:
(1) The overall temperature of a cryopreservation system was reduced to approximately 4 degrees Celsius,
(2) The temperature was first reduced to approximately −4 degrees Celsius at a rate of approximately 1 degree Celsius/minute;
(3) The temperature was then reduced to −55 degrees Celsius at a rate of approximately 20 degrees Celsius/minute;
(4) The temperature was subsequently increased to −20 degrees Celsius at a rate of approximately 10 degrees Celsius/minute;
(5) The temperature was then maintained at approximately −20 degrees Celsius for approximately 3 minutes;
(6) The temperature was then reduced to approximately −40 degrees Celsius at a rate of approximately 1 degree Celsius/minute;
(7) The temperature was then maintained at approximately −40 degrees Celsius for approximately 3 minutes;
(8) Finally, the temperature was reduced to approximately −90 degrees Celsius at a rate of approximately 10 degrees Celsius/minute.

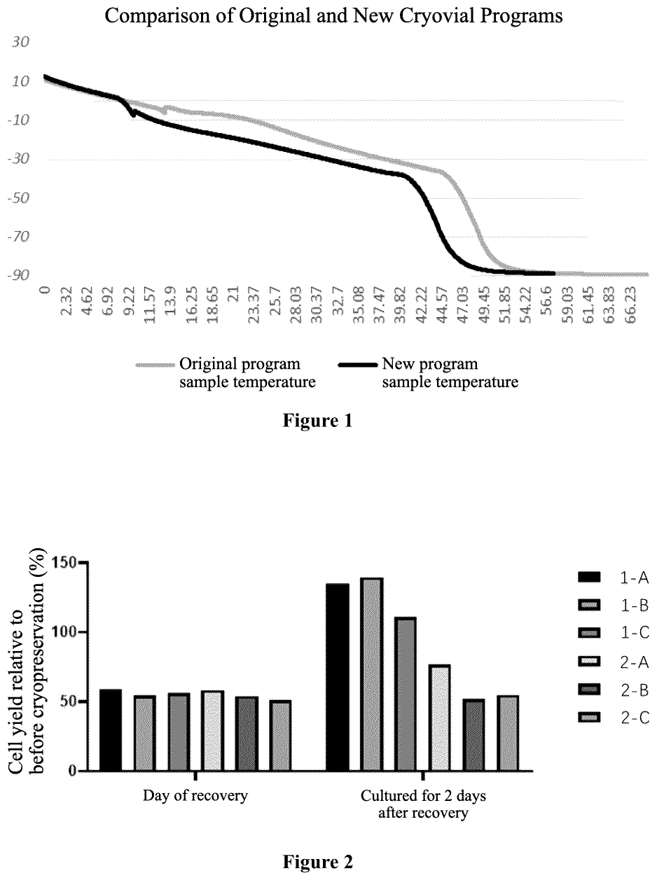

FIG. 1 shows the cooling curve of the cryopreservation program of the present invention. Compared to the cryovial program recommended by the cryopreservation equipment (original program), the cooling curve of the cryopreservation program provided by the present invention (new program) was more balanced, and no temperature rebound was observed. The cryopreservation process of the cryopreservation program of the present invention could be less prone to occurrence of intracellular crystallization.

### Comparison of Effects on Cells Between the Original Program and the Program of the Present Invention

The groups were as follows: Group 1 was cryopreserved using programmable cooling equipment, and Group 2 was cryopreserved using a programmable cooling box. The letters A, B, and C following the group number indicated three different cell cryopreservation densities (2E8/mL, 1E8/mL, 5E7/mL). After TIL cells were resuspended in the cryopreservation solution in the cryovials described in this example and were adjusted to the required densities, the three groups 1-A, 1-B, and 1-C were cryopreserved using the programmable cooling equipment, wherein each step was adopted from the method steps of the present invention. Groups 2-A, 2-B, and 2-C were cryopreserved using the default original program in the programmable cooling box. After programmed cooling, the cells were transferred to liquid nitrogen and recovered one week later.

On day 0 (D0), TIL cells in each group were recovered, and the cryopreservation solution was removed by washing. The cells were counted, and the yield was statistically determined. The cell density was adjusted to 1E6 mL using #5CMI culture medium, and the cells were seeded into 6-well plates with 3~5 mL per well. After two days of culture (culture 2D), the cells were counted again, and the yield was statistically determined. Subsequently, cells were collected for flow cytometry analysis, and tumor cell killing and cytokine secretion were assessed.

FIG. 2 shows the cell counting results obtained from each cooling program. The results indicated that there was no significant difference in the recovery yield among the groups at the time of recovery. After recovery and further culturing for two days, the number of cells in each subgroup of Group 1 had increased, indicating that the cells were expanding. The number of cells in each subgroup of Group 2 was still decreasing, indicating that they were still undergoing apoptosis. The results demonstrated that the cells obtained using the new program provided by the present invention possessed a stronger cell recovery capability.

### Example 2

The groups were as follows: Group 1 was cryopreserved using programmable cooling equipment, and Group 2 was cryopreserved using a programmable cooling box. The letters A, B, and C following the group number indicated three different cell cryopreservation densities (2E8/mL, 1E8/mL, and 5E7/mL). After TIL cells were resuspended in the cryopreservation solution in the cryovials of Example 1 and were adjusted to the required densities, the three groups 1-A, 1-B, and 1-C were cryopreserved using the programmable cooling equipment, wherein each step was adopted from the steps of cryopreservation method 1 of the present invention. Groups 2-A, 2-B, and 2-C were cryopreserved using the default original program in the programmable cooling box. After programmed cooling, the cells were transferred to liquid nitrogen and recovered one week later.

### Flow Cytometry

From each group, 2E5 live cells were taken, washed with PBS, and then stained for surface markers. CD3, CD4, CD8, 7AAD, and Annexin V were labeled, and the proportion of each marker was detected by flow cytometry.

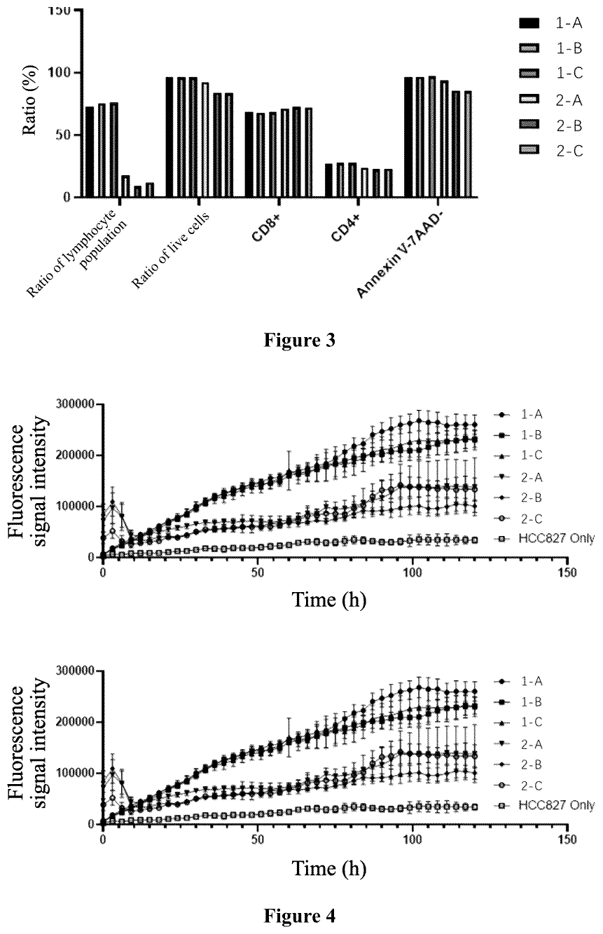

FIG. 3 shows the flow cytometry detection results of cells obtained from each cooling program. The results indicated that, in each subgroup of Group 1, both ratio of lymphocyte population and viability of cells were higher than those of cells in each subgroup of Group 2, and the apoptosis ratio was lower than that of cells in each group of Group 2. There was no difference in CD4 and CD8 ratios among the groups. The results demonstrated that the cells obtained using the new program provided by the present invention possessed a stronger immune cell viability.

### Promotion of Tumor Cell Killing

On day 2(D 2), A375 and Hey T30 tumor cell lines were seeded separately at 2×10⁴ cells/well into two 96-well plates. After 4 hours, TIL cells from each group were co-cultured with the aforementioned tumor cells at an effector-to-target ratio (TIL cells:tumor cells) of 4:1, with 4 wells per group. Caspase-3 substrate was added to the wells, and then the plates were placed in an IncuCyte for scanning. The scanning continued for 5 days. The intensity of the fluorescence signal indicated the occurrence of apoptosis in each well.

FIG. 4 shows the results of the promotion of tumor cell killing of cells obtained from each cooling program. The results showed fluorescence signals at 0 hours originated from the apoptosis of TIL cells themselves and, as the co-culture time extended, increases in fluorescence signals originated from the apoptosis of tumor cells. The results demonstrated that the killing ability of cells in each group of the present invention against the two types of tumor cells was significantly superior to that of cells cryopreserved using the original program of the programmable cooling box. At different cell densities, the tumor cell killing ability of cells in each group of the present invention was significantly superior to that of the original program of the programmable cooling box.

### Promotion of Cytokine Secretion

Twenty-four hours after the aforementioned tumor cell killing experiment was conducted, the 96-well plates were removed from the IncuCyte. Without disturbing the cells, 30 μL of supernatant was collected from each well and placed into a new 96-well plate. The supernatant was cryopreserved at −80° C. for subsequent cytokine detection. After the supernatant was collected, the 96-well plates used for tumor cell killing were returned to their original positions in the IncuCyte to continue scanning.

The cytokine secretion detection method could be performed by referring to the instructions of the cytokine detection kit (BD Biosciences). Human Th1/Th2/Th17 cytokine standard lyophilized powder (BD Biosciences) was reconstituted using 2 mL of Assay Diluent (BD Biosciences) (the concentration of each cytokine in the standard stock solution was 5000 pg/mL), serially diluted in the following order: 1:2, 1:4, 1:8, 1:16, 1:32, 1:64, 1:128, 1:256, 1:512, 1:1024, and labeled as “standard tubes”. One tube containing only Assay Diluent was used as control. Each type of Capture Beads (BD Biosciences) was added at 2 μL/bead/well, and then PE Detection Reagent (BD Biosciences) was added at 10 μL/well and mixed to prepare a mixture (mix). This mixture was added to a V-bottom 96-well plate at 24 μL/well. Subsequently, 10 μL/well of each standard and the supernatant from the experimental groups were added and mixed. The plate was incubated at room temperature in the dark for 3 hours. At the end of the incubation, 200 μL of Wash Buffer (BD Biosciences) was added to each well, and the plate was centrifuged at 500 g for 3 minutes. After centrifugation, the supernatant was discarded, and 100 μL of Wash Buffer (BD Biosciences) was added to each well to resuspend the pellet. Flow cytometry analysis was then performed.

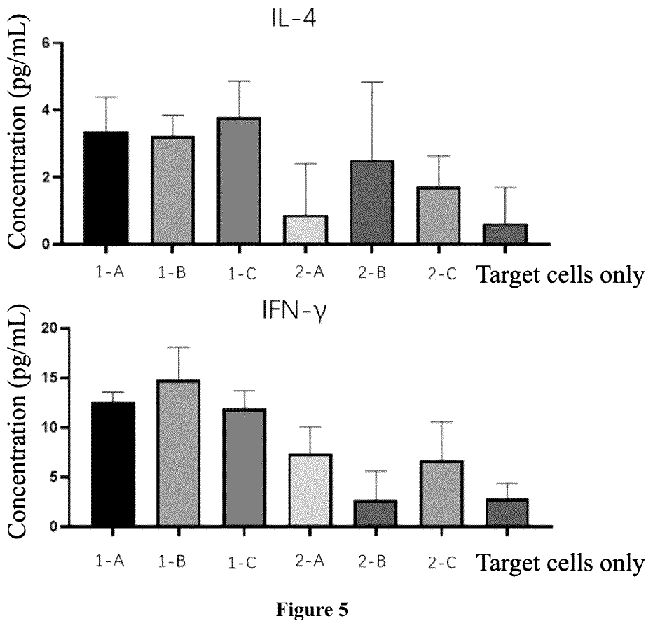

FIG. 5 shows the results of the promotion of cytokine secretion of cells obtained from each cooling program. The results indicated that the secretion amounts of IL-4 and IFN-γ by cells in each group cryopreserved according to the present invention were significantly superior to those of cells cryopreserved using the original program of the programmable cooling box. At different cell densities, the secretion amounts of IL-4 and IFN-γ by cells cryopreserved according to the present invention were significantly superior to those of the original program of the programmable cooling box.

### Example 3

### Using the Cryopreservation Program of the Present Invention for PBMC Cell Cryopreservation

PBMCs were obtained, irradiated with 50~100 Gy X-rays, and then cryopreserved on the same day after irradiation. Cryopreservation was performed using programmable cooling equipment according to the steps of cryopreservation method 1 of the present invention. The cells were preserved in liquid nitrogen. After recovery, cell viability was examined by counting, and the cell yield was calculated based on the cell count results before and after irradiation.

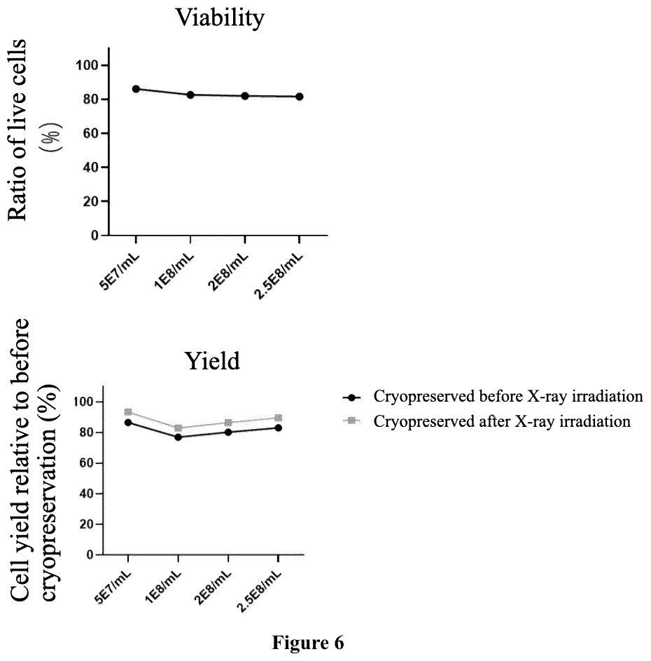

FIG. 6 shows the viability and yield results when the cryopreservation program of the present invention was used for PBMC cell cryopreservation. The results showed that the density of cryopreserved cells was between 5E7/mL and 2.5E8/mL, and that both the cell viability and yield of PBMCs cryopreserved according to the program of the present invention were good after recovery.

After recovery, PBMC cells from each group were used as feeder cells to activate TILs. The activated TILs and feeder cells were mixed and cultured at a TIL:feeder ratio of 1:40~1:400 for 14 days, and the expansion trend of TIL cells was monitored.

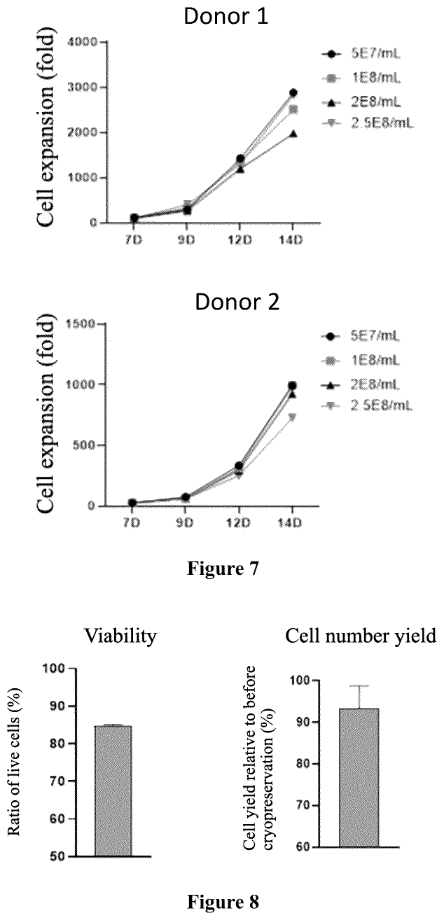

FIG. 7 shows the TIL activation results when the cryopreservation program of the present invention was used for cryopreservation of PBMC cells. The results showed that the cryopreserved cell density ranged from 5E7/mL to 2.5E8/mL, and that each group of cells exhibited a similar enhancing effect on the expansion of TIL cells.

### Example 4

### Using the Steps of Cryopreservation Method 1 of the Present Invention for NK Cell Cryopreservation

Cryopreservation: NK cells were removed from the incubator, mixed well, and counted using an NC250 counter. All cells were transferred to a 50 mL centrifuge tube and centrifuged at 500 g for 5 minutes at room temperature. After centrifugation was completed, all supernatant was discarded, and the cells were resuspended at a concentration of 1E7/mL in the cryopreservation solution in the cryovial of Example 1, which had been pre-chilled to 4° C. The cell suspension was transferred to cryovials with 1 mL/tube, and the cryovials were placed in a cell cryopreservation box pre-chilled to 4 degrees Celsius. The programmable cooling equipment group was transferred to the programmable cooling equipment for cryopreservation, and the cryopreservation box group was cryopreserved at −80° C. in the cryopreservation box. The cells were transferred to a liquid nitrogen tank 24-48 hours after cryopreservation.

Recovery: NK cell cryovials were removed from the liquid nitrogen tank, placed in liquid nitrogen, and transferred to a clean area. The cryovials were thawed in a 37° C. water bath until only soybean-sized ice crystals remained. The cryovials were then sterilized with 70% alcohol and transferred to a biological safety cabinet. The entire cell suspension was added dropwise to 9 mL of NK culture medium pre-warmed to 37° C., mixed well, and counted using an NC250 counter.

FIG. 8 shows the viability and yield results when cryopreservation method 1 of the present invention was used for NK cell cryopreservation. The results showed that both the cell viability and yield of NK cells cryopreserved according to the program of the present invention were good after recovery.

### Example 5

### Using Cryopreservation Method 1 of the Present Invention for TCR-T Cell Cryopreservation

### PBMC (Peripheral Blood Mononuclear Cell) Isolation

Apheresis blood was transferred to 50 mL centrifuge tubes, washed and combined with PBS or normal saline, and centrifuged at 3000 g for 10 minutes. After centrifugation, the supernatant plasma was discarded. The cell pellet was diluted with PBS or normal saline, and the diluted blood cell mixture was slowly added to the supernatant of 20 mL of lymphocyte separation medium (Tianjin Haoyang).

Centrifugation was performed using a horizontal rotor at 500-600 g for 15-30 minutes at a temperature of 18-22° C. After centrifugation, supernatant plasma and normal saline were aspirated and discarded, and the middle buffy coat layer was transferred to another clean 50 mL centrifuge tube. The collected buffy coat layer was diluted with PBS or normal saline and centrifuged at 600 g for 10 minutes at room temperature. After centrifugation, the supernatant was discarded, and the cells were washed once with PBS or normal saline and centrifuged at 500 g for 5 minutes at room temperature.

### T Cell Isolation

The separated PBMCs were resuspended in PBS solution containing 2% FBS and 1 mM EDTA. T cells were isolated using an Easy Sep Human T Cell Isolation Kit (Stemcell). The isolated cells were centrifuged at 500 g for 5 minutes, resuspended in cryopreservation solution, and placed in a programmable cooling box for cryopreservation in liquid nitrogen.

### T Cell Activation and Transduction

T cells cryopreserved in liquid nitrogen were recovered and cultured, centrifuged and resuspended in T cell culture medium RPMI1640 (Gibco)+10% FBS (Bovogen) to a density of 5E5/mL. T cell TransAct (Miltenyi) was added at a ratio of 1:100, and recombinant human IL-2 was added at a concentration of 30 IU/mL. The cells were cultured for approximately 72 hours.

One day before transduction, 24-well suspension culture plates were coated with recombinant human fibronectin fragment (Retronectin, Takara) at a final concentration of 15 μg/mL, with 250 μL per well of the 24-well plate. The plates were protected from light and stored overnight at 4° C. for later use. The coated 24-well plates were taken out, the coating solution was aspirated and discarded, and 500 μL of blocking solution containing 2% BSA was added for blocking at room temperature for 30 minutes. The blocking solution was aspirated and discarded, the plates were washed twice with 500 μL/well of washing solution containing 2.5% HEPES, and the washing solution was aspirated and discarded. The experimental group was transduced with a retrovirus carrying a specific TCR nucleic acid fragment of the NY-ESO-1 antigen peptide (SLLMWITQC).

Retrovirus solution (0.1~1 mL) was added to each well, and the plate was centrifuged at 32° C. and 2000 g for 2 hours. The supernatant from the 24-well plate was discarded. Recovered and activated T cells were added to each well of the 24-well plate, with a volume of 500-1000 μL and a cell concentration of approximately 5×10⁵ cells/mL. The plate was centrifuged at 30-32° C. and 1000 g for 10 minutes. After centrifugation, the culture plate was placed in a 37° C., 5% CO 2 incubator for culturing to obtain transduced cells. After transduction, the cells were cultured for approximately 0-4 days to obtain TCR-T cell populations.

### Culture After TCR-T Transduction

After transduction, based on the cell count density and viability determined every 1-3 days, T cell culture medium was added according to the count results. Recombinant human IL-2 was added at a concentration of 30~100 IU/mL, and the initial culture cell density was adjusted to 0.5~2×10⁶ cells/mL. The cells were cultured for 5~10 days.

Before and after programmed cell cryopreservation, sampled cells were stained with AO/DAPI (ChemoMetec) to calculate cell viability.

After programmed cell cryopreservation, cells were recovered and cultured in T cell culture medium. Recombinant human IL-2 was added at a concentration of 100 IU/mL, the cell density was adjusted to 1~2×10⁶ cells/mL, and the cells were cultured for 3 days. Sampled cells were stained with AO/DAPI (ChemoMetec) to calculate cell viability.

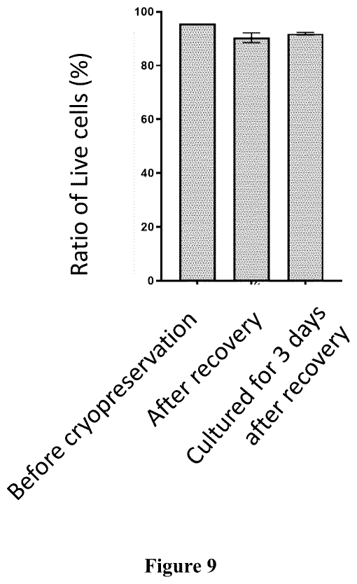

FIG. 9 shows the viability results when cryopreservation method 1 of the present invention was used for TCR-T cell cryopreservation. The results showed that the TCR-T cells cryopreserved according to the program of the present invention had good cell viability after recovery.

After programmed cell cryopreservation, cells were recovered and cultured in T cell culture medium. Recombinant human IL-2 was added at a concentration of 100 IU/mL, the cell density was adjusted to 1~2×10⁶ cells/mL, and the cells were cultured for 3 days.

A 375-GFP was seeded into a 96-well plate at 2E4 cells/well one day in advance. TCR-T cells cultured for 3 days were collected and resuspended in fresh T cell culture medium. The cells were added to each well at concentrations of 2E4/well, 1E4/well, and 5E3/well. Super View 488 Caspase-3 dye (UElandy) was added, and the plate was placed in an IncuCyte for live cell analysis.

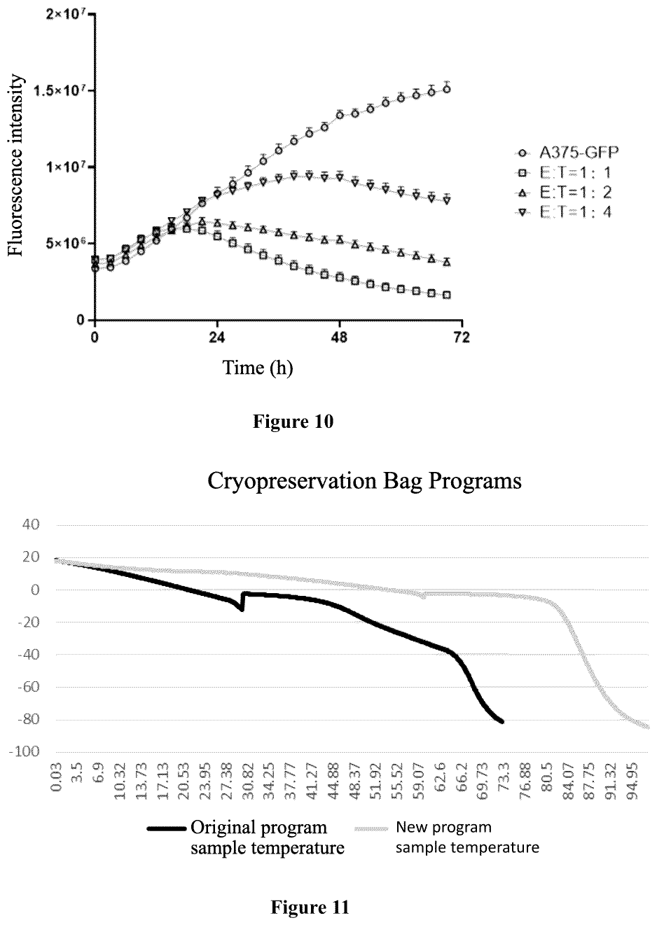

FIG. 10 shows the tumor cell killing results of TCR-T cells when cryopreservation method 1 of the present invention was used. The results showed that the tumor cell killing effect of cells in each group was similarly enhanced.

### Example 6

### Cell Cryopreservation

The cryopreservation bag program recommended by the cryopreservation equipment employed (Thermo Scientific) (original program) and the cryopreservation program provided by the present invention (new program) were used for the cryopreservation of cells cryopreserved in the present invention. The main cryopreservation components within the cryopreservation system were: a DMSO-comprising ready-to-use cryopreservation solution (10~50%), a 20% HAS injection (2~25%), and a compound electrolyte injection (25-88%).

The cooling process of cryopreservation method 2 of the present invention was as follows:
(1) The overall temperature of a cryopreservation system was reduced to approximately 10 degrees Celsius,
(2) The temperature was first reduced to approximately 5 degrees Celsius at a rate of approximately 1 degree Celsius/minute;
(3) The temperature was then maintained at approximately 5 degrees Celsius for approximately 3 minutes;
(4) The temperature was then reduced to approximately −2 degrees Celsius at a rate of approximately 0.5 degrees Celsius/minute;
(5) The temperature was then reduced to approximately −60 degrees Celsius at a rate of approximately 35 degrees Celsius/minute;
(6) The temperature was then increased to approximately −10 degrees Celsius at a rate of approximately 30 degrees Celsius/minute;
(7) The temperature was then reduced to approximately −15 degrees Celsius at a rate of approximately 3 degrees Celsius/minute;
(8) The temperature was then maintained at approximately −15 degrees Celsius for approximately 5 minutes;
(9) The temperature was then reduced to approximately −35 degrees Celsius at a rate of approximately 2 degrees Celsius/minute;
(10) Subsequently, the temperature was reduced to approximately −90 degrees Celsius at a rate of approximately 15 degrees Celsius/minute;
(11) Finally, the temperature was maintained at approximately −90 degrees Celsius for approximately 10 minutes.

FIG. 11 shows the cooling curve of cryopreservation method 2 of the present invention. Compared to the cryopreservation bag program recommended by the cryopreservation equipment (original program), the cooling curve of cryopreservation method 2 provided by the present invention (new program) was more balanced, and no obvious temperature rebound was observed. The cryopreservation process of the cryopreservation program of the present invention could be less prone to occurrence of intracellular crystallization.

For example, the cryopreservation bag program recommended by the cryopreservation equipment (original program) involved reducing the overall temperature of the cryopreservation system to 20 degrees Celsius, then to −6 degrees Celsius at 1 degree Celsius/minute, then to −50 degrees Celsius at 25 degrees Celsius/minute, then to −14 degrees Celsius at 10 degrees Celsius/minute, then to −45 degrees Celsius at 1 degree Celsius/minute, and finally to −90 degrees Celsius at 10 degrees Celsius/minute.

Cryopreserved cell samples from each group were packaged in 2 mL cryovials, 50 mL cryopreservation bags, and 750 mL cryopreservation bags, respectively, and then cryopreserved. After programmed cooling, the cells were transferred to liquid nitrogen and recovered one week later. A 2 mL cryovial contained 1 mL of cell suspension, a 50 mL cryopreservation bag contained 10 mL of cell suspension, and a 750 mL cryopreservation bag contained 90 mL of cell suspension. The cell density in each group was consistent.

### Cell Recovery and Culture

On day 0 (D0), after TIL cells in each group were recovered and the cryopreservation solution was removed by washing, the cells were counted, and the yield was statistically determined. At the same time, cells were collected for flow cytometry detection. Some cells were taken, and the cell density was adjusted to 1E6/mL using commercial T cell culture media, such as those from Stem Cell, Lonza, Thermo, Miltenyi, and other brands. The cells were seeded into 6-well plates with 3~5 mL per well. After culturing for 2~4 days, cells were collected for tumor cell killing detection.

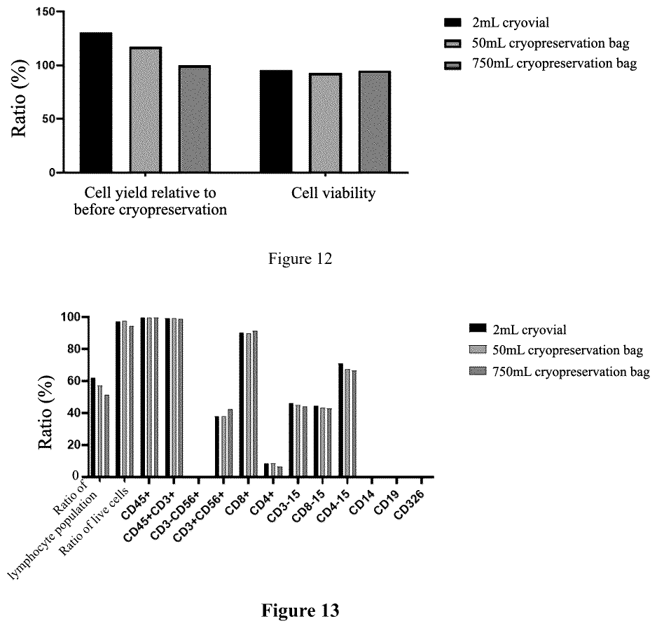

FIG. 12 shows the cell count results obtained in each cryopreservation environment using cryopreservation method 2. The left panel shows the recovery ratio of cell number, and the right panel shows the recovery ratio of cell viability. The results indicated that both the cell yield and viability ratio in each group were high during recovery following cryopreservation method 2.

### Example 7

### Flow Cytometry

From each group, 2E5 live cells were taken, washed with PBS, and then stained for surface markers. CD45, CD3, CD4, CD8, Viability, IL15Ra, CD14, CD19, and CD326 were labeled, and the proportion of each marker was detected by flow cytometry.

FIG. 13 shows the flow cytometry detection results of cells obtained in various cryopreservation environments using cryopreservation method 2. The results indicated that when cryopreservation method 2 provided by the present invention was used for each group of cells in different types of cryovials and cryopreservation bags, there was no difference in the proportion of each marker in the cells, and all groups exhibited a high proportion of live cells.

### Promotion of Tumor Cell Killing

On day 4(D4), A375
tumor cell lines were seeded at 2×10⁴ cells/well into two 96-well plates. After 4 hours, TIL cells from each group were co-cultured with the aforementioned tumor cells at an effector-to-target ratio (TIL cells: tumor cells) of 4:1, with 4 wells per group. Caspase-3 substrate was added to the wells, and then the plates were placed in an IncuCyte for scanning. The scanning continued for 5 days. The intensity of the fluorescence signal indicated the occurrence of apoptosis in each well.

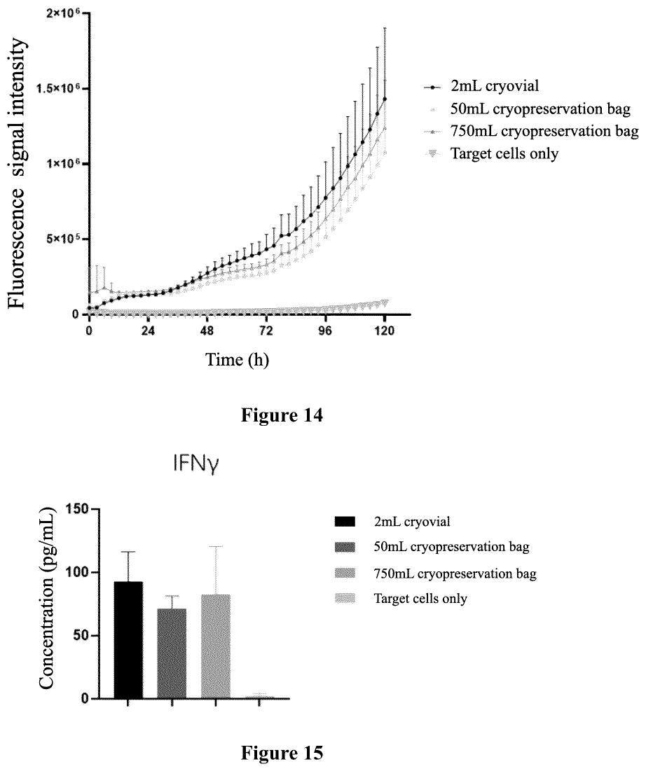

FIG. 14 shows the results of the promotion of tumor cell killing of cells obtained in each cryopreservation environment using cryopreservation method 2. The results showed fluorescence signals at 0 hours originated from the apoptosis of TIL cells themselves and, as the co-culture time extended, increases in fluorescence signals originated from the apoptosis of tumor cells. The results demonstrated that when cryopreservation method 2 provided by the present invention was used for each group of cells in different types of cryovials and cryopreservation bags, each group of cells possessed killing ability against tumor cells.

### Promotion of Cytokine Secretion

Twenty-four hours after the aforementioned tumor cell killing experiment was conducted, the 96-well plates were removed from the IncuCyte. Without disturbing the cells, 30 μL of supernatant was collected from each well and placed into a new 96-well plate. The supernatant was cryopreserved at −80° C. for subsequent cytokine detection. After the supernatant was collected, the 96-well plates used for tumor cell killing were returned to their original positions in the IncuCyte to continue scanning.

The cytokine secretion detection method could be performed by referring to the instructions of the cytokine detection kit (BD Biosciences). Human Th1/Th2/Th17 cytokine standard lyophilized powder (BD Biosciences) was reconstituted using 2 mL of Assay Diluent (BD Biosciences) (the concentration of each cytokine in the standard stock solution was 5000 pg/mL), serially diluted in the following order: 1:2, 1:4, 1:8, 1:16, 1:32, 1:64, 1:128, 1:256, 1:512, 1:1024, and labeled as “standard tubes”. One tube containing only Assay Diluent was used as control. Each type of Capture Beads (BD Biosciences) was added at 2 μL/bead/well, and then PE Detection Reagent (BD Biosciences) was added at 10 μL/well and mixed to prepare a mixture (mix). This mixture was added to a V-bottom 96-well plate at 24 μL/well. Subsequently, 10 μL/well of each standard and the supernatant from the experimental groups were added and mixed. The plate was incubated at room temperature in the dark for 3 hours. At the end of the incubation, 200 μL of Wash Buffer (BD Biosciences) was added to each well, and the plate was centrifuged at 500 g for 3 minutes. After centrifugation, the supernatant was discarded, and 100 μL of Wash Buffer (BD Biosciences) was added to each well to resuspend the pellet. Flow cytometry analysis was then performed.

FIG. 15 shows the results of the promotion of cytokine secretion of cells obtained in each cryopreservation environment using cryopreservation method 2. The results indicated that when cryopreservation method 2 provided by the present invention was used for each group of cells in different types of cryovials and cryopreservation bags, each group of cells possessed the ability to release cytokines.

### Example 8. Using the Cryopreservation Program of the Present Invention for Cryopreservation of Different Types of Cells

PBMCs were obtained and cryopreserved in the programmable cooling equipment of cryopreservation method 2 of the present invention and preserved in liquid nitrogen. After recovery, cell viability was measured by counting, and cell yield was calculated based on the cell count results before and after irradiation. Furthermore, NK cells could be removed from the incubator, cryopreserved in the programmable cooling equipment of the present invention, and preserved in liquid nitrogen. After recovery, cell viability was examined by counting.

Additionally, T cells could be transduced with a retrovirus carrying a specific TCR nucleic acid fragment of the NY-ESO-1 antigen peptide to obtain TCR-T cell populations; subsequently, these cells were cryopreserved in the programmable cooling equipment of cryopreservation method 2 of the present invention, preserved in liquid nitrogen, and after recovery, cell viability was examined by counting.

The results showed that when cryopreservation method 2 of the present invention was used to cryopreserve different types of cells, the cell viability after recovery was good.

### Example 9. Other Preferred Parameters for Cryopreservation Methods

### (I) Cryopreservation Methods 1-1 to 1-3

### Cryopreservation Method 1-1

(1) The overall temperature of a cryopreservation system was reduced to approximately 4 degrees Celsius,
(2) The temperature was first reduced to approximately −2° C. at a rate of approximately 1.5° C./min;
(3) The temperature was then reduced to −60 degrees Celsius at a rate of approximately 15° C./min;
(4) The temperature was subsequently increased to −20 degrees Celsius at a rate of approximately 15° C./min;
(5) The temperature was then maintained at the end temperature of the previous step for approximately 5 min;
(6) The temperature was then reduced to approximately −45 degrees Celsius at a rate of approximately 1.5° C./min;
(7) The temperature was then maintained at the end temperature of the previous step for approximately 5 min;
(8) Finally, the temperature was reduced to approximately −80 degrees Celsius at a rate of approximately 15° C./min.

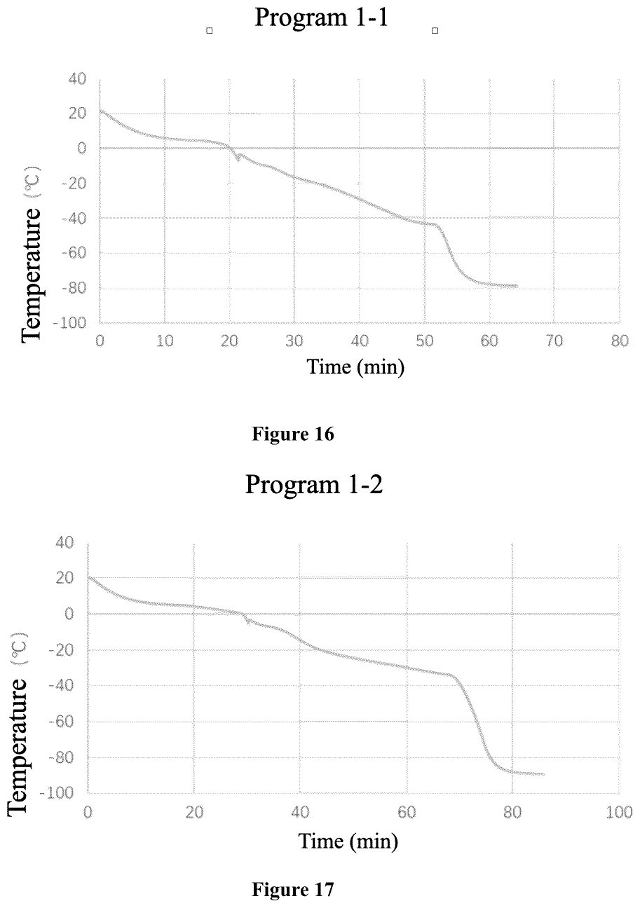

FIG. 16 shows the cooling curve of cryopreservation method 1-1 of the present invention. The cooling curve of cryopreservation method 1-1 provided by the present invention was balanced, and no obvious temperature rebound was observed. The cryopreservation process of the cryopreservation program of the present invention could be less prone to occurrence of intracellular crystallization.

### Cryopreservation Method 1-2

(1) The overall temperature of a cryopreservation system was reduced to approximately 4 degrees Celsius,
(2) The temperature was first reduced to approximately −2° C. at a rate of approximately 0.5° C./min;
(3) The temperature was then reduced to −50 degrees Celsius at a rate of approximately 25° C./min;
(4) The temperature was subsequently increased to −20 degrees Celsius at a rate of approximately 10° C./min;
(5) The temperature was then maintained at the end temperature of the previous step for approximately 2 min;
(6) The temperature was then reduced to approximately −35 degrees Celsius at a rate of approximately 0.5° C./min;
(7) The temperature was then maintained at the end temperature of the previous step for approximately 2 min;
(8) Finally, the temperature was reduced to approximately −90 degrees Celsius at a rate of approximately 8° C./min.

FIG. 17 shows the cooling curve of cryopreservation method 1-2 of the present invention. The cooling curve of cryopreservation method 1-2 provided by the present invention was balanced, and no obvious temperature rebound was observed. The cryopreservation process of the cryopreservation program of the present invention could be less prone to occurrence of intracellular crystallization.

### Cryopreservation Method 1-3

(1) The overall temperature of a cryopreservation system was reduced to approximately 4 degrees Celsius,
(2) The temperature was first reduced to approximately −4° C. at a rate of approximately 1° C./min;
(3) The temperature was then reduced to −55 degrees Celsius at a rate of approximately 20° C./min;
(4) The temperature was subsequently increased to −18 degrees Celsius at a rate of approximately 10° C./min;
(5) The temperature was then maintained at the end temperature of the previous step for approximately 3 min;
(6) The temperature was then reduced to approximately −45 degrees Celsius at a rate of approximately 1.5° C./min;
(7) The temperature was then maintained at the end temperature of the previous step for approximately 5 min;
(8) Finally, the temperature was reduced to approximately −90 degrees Celsius at a rate of approximately 15° C./min.

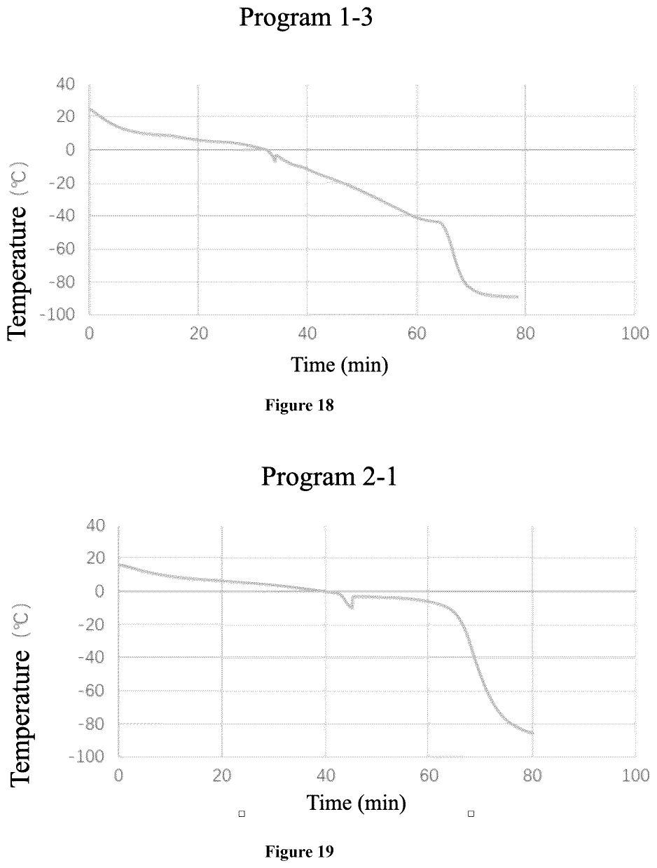

FIG. 18 shows the cooling curve of cryopreservation method 1-3 of the present invention. The cooling curve of cryopreservation method 1-3 provided by the present invention was balanced, and no obvious temperature rebound was observed. The cryopreservation process of the cryopreservation program of the present invention could be less prone to occurrence of intracellular crystallization.

### (II) Cryopreservation Methods 2-1 to 2-2

### Cryopreservation Method 2-1

(1) The overall temperature of a cryopreservation system was reduced to approximately 5° C.
(2) The temperature was reduced to approximately 3° C., where the rate of reduction was approximately 0.5° C./min
(3) The temperature was maintained at approximately 3° C. for approximately 3 minutes;
(4) The temperature was reduced to approximately −1 degree Celsius, where the rate of reduction was approximately 0.5 degrees Celsius/min;
(5) The temperature was reduced to approximately −55 degrees Celsius, where the rate of reduction was approximately 30 degrees Celsius/min;
(6) The temperature was increased to approximately −10 degrees Celsius, where the rate of increase was 25° C./min;
(7) The temperature was reduced to approximately −13 degrees Celsius, where the rate of reduction was approximately 2° C./min;
(8) The temperature was maintained at approximately −13 degrees Celsius for approximately 3 minutes;
(9) The temperature was reduced to approximately −40 degrees Celsius, where the rate of reduction was approximately 2° C./min;
(10) The temperature was reduced to approximately −90 degrees Celsius, where the rate of reduction was approximately 10° C./min.
(11) Finally, the temperature was maintained at approximately −90 degrees Celsius for approximately 10 minutes.

FIG. 19 shows the cooling curve of cryopreservation method 2-1 of the present invention. The cooling curve of cryopreservation method 2-1 provided by the present invention was balanced, and no obvious temperature rebound was observed. The cryopreservation process of the cryopreservation program of the present invention could be less prone to occurrence of intracellular crystallization.

### Cryopreservation Method 2-2

(1) The overall temperature of a cryopreservation system was reduced to approximately 15°° C.
(2) The temperature was reduced to approximately 4° C., where the rate of reduction was approximately 1° C./min
(3) The temperature was maintained at approximately 4° C. for approximately 3 minutes;
(4) The temperature was reduced to approximately −4 degrees Celsius, where the rate of reduction was approximately 1.5 degrees Celsius/min;
(5) The temperature was reduced to approximately −50 degrees Celsius, where the rate of reduction was approximately 25 degrees Celsius/min;
(6) The temperature was increased to approximately −8 degrees Celsius, where the rate of increase was 35° C./min;
(7) The temperature was reduced to approximately −15 degrees Celsius, where the rate of reduction was approximately 1.5° C./min;
(8) The temperature was maintained at approximately −15 degrees Celsius for approximately 3 minutes;
(9) The temperature was reduced to approximately −45 degrees Celsius, where the rate of reduction was approximately 2° C./min;
(10) The temperature was reduced to approximately −90 degrees Celsius, where the rate of reduction was approximately 20° C./min;
(11) Finally, the temperature was maintained at approximately −90 degrees Celsius for approximately 10 minutes.

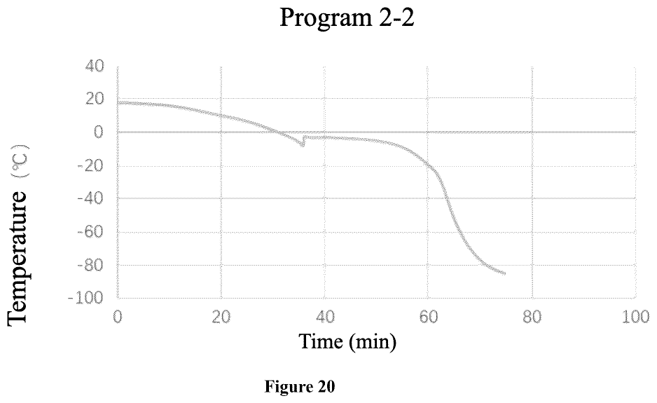

FIG. 20 shows the cooling curve of cryopreservation method 2-2 of the present invention. The cooling curve of cryopreservation method 2-2 provided by the present invention was balanced, and no obvious temperature rebound was observed. The cryopreservation process of the cryopreservation program of the present invention could be less prone to occurrence of intracellular crystallization.

The foregoing detailed description was provided through explanation and examples, and was not intended to limit the scope of the appended claims. Various modifications to the presently listed embodiments of the invention are apparent to those skilled in the art and remain within the scope of the appended claims and their equivalents.

1. A cryopreservation method, comprising the steps of:
(1) reducing the overall temperature of a cryopreservation system to approximately 4 to 15 degrees Celsius;
(2) first, reducing the temperature to approximately −2~−6 degrees Celsius at a rate of approximately 0.5~1.5 degrees Celsius/minute;
(3) then, reducing the temperature to −50~−60 degrees Celsius at a rate of approximately 15~25 degrees Celsius/minute;
(4) then, increasing the temperature to −15~−25 degrees Celsius at a rate of approximately 8~15 degrees Celsius/minute;
(5) then, maintaining the end temperature of the preceding step for approximately 2~5 minutes;
(6) then, reducing the temperature to approximately −35~−45 degrees Celsius at a rate of approximately 0.5~1.5 degrees Celsius/minute;
(7) then, maintaining the end temperature of the preceding step for approximately 2~5 minutes; and
(8) finally, reducing the temperature to approximately −80~−90 degrees Celsius at a rate of approximately 8~15 degrees Celsius/minute.

2. The method of claim 1, wherein the method further comprises, before cooling the cryopreservation system in step (1), mixing a sample comprising cells with a cryopreservation agent at a volume ratio of approximately 1:0.5~1:10, wherein the cryopreservation agent comprises: 10~50% (w/v) of CS 10, and 2~25%% (w/v) of 20% human serum albumin injection.

3. The method of any one of claims 1-2, wherein the method further comprises, before cooling the cryopreservation system, mixing a sample comprising cells with a culture medium at a volume ratio of approximately 1:0.5~1:10, wherein the culture medium comprises: 5~50% of FBS, and 10~90% of a basal medium (e.g., 1640 or DMEM).

4. The method of any one of claims 1-3, wherein the method further comprises, after cooling the cryopreservation system in step (8), storing the cryopreservation system at approximately −80 degrees Celsius or below.

5. The method of any one of claims 1-4, wherein the method further comprises, after cooling the cryopreservation system in step (8), storing the cryopreservation system in liquid nitrogen.

6. The method of any one of claims 1-5, wherein the cryopreservation system comprises the sample placed in a cryovial.

7. The method of any one of claims 1-6, wherein the volume of the cryopreservation system is from approximately 100 microliters to 5 mL.

8. The method of any one of claims 1-7, wherein the cryopreservation system comprises cells.

9. The method of claim 8, wherein the density of the cells in the cryopreservation system is about 1×10⁵ to 1×10⁹ cells/mL.

10. The method of any one of claims 8-9, wherein the cells comprise immune cells.

11. The method of any one of claims 8-10, wherein the cells comprise phagocytes, lymphocytes, neutrophils, eosinophils, and/or basophils.

12. The method of any one of claims 8-11, wherein the cells comprise monocytes, macrophages, and/or dendritic cells.

13. The method of any one of claims 8-12, wherein the cells comprise B cells, T cells, natural killer cells, and/or natural killer-like T cells.

14. The method of any one of claims 8-13, wherein the cells comprise αβ T cells and/or γδ T cells.

15. The method of any one of claims 8-14, wherein the cells comprise tumor-infiltrating lymphocytes (TILs).

16. The method of claim 15, wherein the TILs are derived from tumor tissue fragments, pleural effusion, and/or peritoneal effusion, and/or are TILs recovered after cryopreservation.

17. The method of claim 16, wherein the volume of the fragments is from approximately 1 cubic millimeter to approximately 27 cubic millimeters.

18. A cell, wherein the cell is cryopreserved by the method of any one of claims 1-17.

19. A pharmaceutical composition, comprising the cell of claim 18, and optionally a pharmaceutically acceptable carrier.

20. A method of affecting tumor cell growth, comprising administering the cell of claim 18 and/or the pharmaceutical composition of claim 19.

21. Use of the cell of claim 18 and/or the pharmaceutical composition of claim 19 in the manufacture of a medicament for preventing, alleviating, and/or treating a disease, preferably a tumor.

22. A medicament comprising the cell of claim 18 and/or the pharmaceutical composition of claim 19, for use in preventing and/or treating a disease and/or a symptom, preferably a tumor.

23. A method of preventing and/or treating a disease and/or a symptom, preferably a tumor, comprising administering to a subject in need thereof the cell of claim 18 and/or the pharmaceutical composition of claim 19.

24. The cell of claim 18 and/or the pharmaceutical composition of claim 19, for use in preventing and/or treating a disease and/or a symptom, preferably a tumor.

25. A system, the system comprising equipment configured to perform the method of any one of claims 1-17.
